(19) United States
(12) Patent Application Publication
(10) Pub. No.: US 2025/0002576 A1
(43) Pub. Date: Jan. 2, 2025

(54) CLAUDIN 18.2-BINDING ANTIBODIES AND FRAGMENTS THEREOF

(71) Applicant: Fusion Pharmaceuticals Inc., Hamilton (CA)

(72) Inventors: Philipp Kuhn, Bremen (DE); André Frenzel, Braunschweig (DE)

(21) Appl. No.: 18/703,993

(22) PCT Filed: Oct. 25, 2022
(86) PCT No.: PCT/CA2022/051572

§ 371 Date: Apr. 23, 2024

Related U.S. Application Data
(60) Provisional application No. 63/271,633, filed on Oct. 25, 2021.

(51) Int. Cl. C07K 16/28; G01N 33/68

(52) U.S. Cl. CPC C07K 16/28; G01N 33/6854; C07K 2317/33; C07K 2317/77; C07K 2317/92; C07K 2317/94; G01N 2333/705

(57) ABSTRACT

Disclosed herein are antibodies and antibody fragments capable of binding to claudin 18.2.

### RELATED APPLICATIONS

The present application claims priority to U.S. Provisional Patent Application No. 63/271,633, filed Oct. 25, 2021 the entire contents of which are hereby incorporated by reference for all purposes.

### SEQUENCE LISTING

The instant application contains a Sequence Listing which has been submitted electronically in XML format and is hereby incorporated by reference in its entirety. Said XML file, created on Oct. 24, 2022, is named 20221024_FPI_028WO.xml and is 99 kilobytes in size.

### BACKGROUND

Stomach, esophageal (gastroesophageal: GE) and pancreatic cancers are malignancies with highest unmet medical need. Gastric cancer is the second leading cause of cancer death worldwide while pancreatic cancer is currently fourth leading cause of cancer-related deaths in United States. The majority of patients with gastric cancer are often diagnosed in the advanced stage of the disease, and treatment mostly entails palliative chemotherapy conferring median PFS (progression free survival) of 5 to 7 months and median OS (overall survival) of 9 to 11 months at best. On the other hand, pancreatic cancer has extremely poor prognosis with only about 9% of 5-year survival rate.

Claudins are a family of tight junction proteins with Claudin 18 splice variant 2 (Claudin 18.2 (CLDN18.2)) being one of its members. Claudins mainly form paracellular barrier and pores and regulate the transport of substances by determining the permeability of the tight junctions. The expression of CLDN18.2 is not detectable in normal healthy human tissues except for stomach. However, it is aberrantly expressed at significant levels in about 60-90% of gastroesophageal cancers as well as its metastases and pancreatic cancer making it an attractive therapeutic target. Currently, chimeric IgG1 antibody, IMAB362, directed against CLDN18.2 is in Phase III clinical trial in combination with mFOLFOX6 (NCT03504397) and CAPOX (NCT03653507) chemotherapy in patients with locally advanced unresectable or metastatic gastric and gastroesophageal (GEJ) cancer.

There remains a need for improved antibodies that may be used as therapeutics or part of therapeutics (e.g., cancer therapeutics) that can target CLDN18.2.

### SUMMARY

The present disclosure provided antibodies and antigen-binding fragments that are capable of binding to Claudin 18.2.

In one aspect, provided are antibodies or antigen binding fragments thereof that are capable of binding to Claudin 18.2, comprising:
(a) (i) a heavy chain variable domain (VH) comprising complementarity-determining regions: CDR-H1 comprising the amino acid sequence of SEQ ID NO: 2 or a sequence differing in 1 or 2 amino acids therefrom, CDR-H2 comprising the amino acid sequence of SEQ ID NO: 3 or a sequence differing in 1 or 2 amino acids therefrom, and CDR-H3 comprising the amino acid sequence of SEQ ID NO: 4 or a sequence differing in 1 or 2 amino acids therefrom: and
   (ii) a light chain variable domain (VL) comprising complementarity-determining regions: CDR-L1 comprising the amino acid sequence of SEQ ID NO: 6 or a sequence differing in 1 or 2 amino acids therefrom, CDR-L2 comprising the amino acid sequence of SEQ ID NO: 7 or a sequence differing in 1 or 2 amino acids therefrom, and CDR-L3 comprising the amino acid sequence of SEQ ID NO: 8 or a sequence differing in 1 or 2 amino acids therefrom:
(b) (i) a VH comprising complementarity-determining regions: CDR-H1 comprising the amino acid sequence of SEQ ID NO: 10 or a sequence differing in 1 or 2 amino acids therefrom, CDR-H2 comprising the amino acid sequence of SEQ ID NO: 11 or a sequence differing in 1 or 2 amino acids therefrom, and CDR-H3 comprising the amino acid sequence of SEQ ID NO: 12 or a sequence differing in 1 or 2 amino acids therefrom; and
   (ii) a VL comprising complementarity-determining regions: CDR-L1 comprising the amino acid sequence of SEQ ID NO: 14 or a sequence differing in 1 or 2 amino acids therefrom, CDR-L2 comprising the amino acid sequence of SEQ ID NO: 15 or a sequence differing in 1 or 2 amino acids therefrom, and CDR-L3 comprising the amino acid sequence of SEQ ID NO: 16 or a sequence differing in 1 or 2 amino acids therefrom:
(c) (i) a VH comprising complementarity-determining regions: CDR-H1 comprising the amino acid sequence of SEQ ID NO: 18 or a sequence differing in 1 or 2 amino acids therefrom, CDR-H2 comprising the amino acid sequence of SEQ ID NO: 19 or a sequence differing in 1 or 2 amino acids therefrom, and CDR-H3 comprising the amino acid sequence of SEQ ID NO: 20 or a sequence differing in 1 or 2 amino acids therefrom: and
   (ii) a VL comprising complementarity-determining regions: CDR-L1 comprising the amino acid sequence of SEQ ID NO: 22 or a sequence differing in 1 or 2 amino acids therefrom, CDR-L2 comprising the amino acid sequence of SEQ ID NO: 23 or a sequence differing in 1 or 2 amino acids therefrom, and CDR-L3 comprising the amino acid sequence of SEQ ID NO: 24 or a sequence differing in 1 or 2 amino acids therefrom:
(d) (i) a VH comprising complementarity-determining regions: CDR-H1 comprising the amino acid sequence of SEQ ID NO: 26 or a sequence differing in 1 or 2 amino acids therefrom, CDR-H2 comprising the amino acid sequence of SEQ ID NO: 27 or a sequence differing in 1 or 2 amino acids therefrom, and CDR-H3 comprising the amino acid sequence of SEQ ID NO: 28 or a sequence differing in 1 or 2 amino acids therefrom: and
   (ii) a VL comprising complementarity-determining regions: CDR-L1 comprising the amino acid sequence of SEQ ID NO: 30 or a sequence differing in 1 or 2 amino acids therefrom, CDR-L2 comprising the amino acid sequence of SEQ ID NO: 31 or a sequence differing in 1 or 2 amino acids therefrom, and CDR-L3 comprising the amino acid sequence of SEQ ID NO: 32 or a sequence differing in 1 or 2 amino acids therefrom:
(e) (i) a VH comprising complementarity-determining regions: CDR-H1 comprising the amino acid sequence of SEQ ID NO: 34 or a sequence differing in 1 or 2 amino acids therefrom, CDR-H2 comprising the amino acid sequence of SEQ ID NO: 35 or a sequence differing in 1 or 2 amino acids therefrom, and CDR-H3 comprising the amino acid sequence of SEQ ID NO: 36 or a sequence differing in 1 or 2 amino acids therefrom; and
   (ii) a VL comprising complementarity-determining regions: CDR-L1 comprising the amino acid sequence of SEQ ID NO: 38 or a sequence differing in 1 or 2 amino acids therefrom, CDR-L2 comprising the amino acid sequence of SEQ ID NO: 39 or a sequence differing in 1 or 2 amino acids therefrom, and CDR-L3 comprising the amino acid sequence of SEQ ID NO: 40 or a sequence differing in 1 or 2 amino acids therefrom:
(f) (i) a VH comprising complementarity-determining regions: CDR-H1 comprising the amino acid sequence of SEQ ID NO: 42 or a sequence differing in 1 or 2 amino acids therefrom, CDR-H2 comprising the amino acid sequence of SEQ ID NO: 43 or a sequence differing in 1 or 2 amino acids therefrom, and CDR-H3 comprising the amino acid sequence of SEQ ID NO: 44 or a sequence differing in 1 or 2 amino acids therefrom; and
   (ii) a VL comprising complementarity-determining regions: CDR-L1 comprising the amino acid sequence of SEQ ID NO: 46 or a sequence differing in 1 or 2 amino acids therefrom, CDR-L2 comprising the amino acid sequence of SEQ ID NO: 47 or a sequence differing in 1 or 2 amino acids therefrom, and CDR-L3 comprising the amino acid sequence of SEQ ID NO: 48 or a sequence differing in 1 or 2 amino acids therefrom:
(g) (i) a VH comprising complementarity-determining regions: CDR-H1 comprising the amino acid sequence of SEQ ID NO: 50 or a sequence differing in 1 or 2 amino acids therefrom, CDR-H2 comprising the amino acid sequence of SEQ ID NO: 51 or a sequence differing in 1 or 2 amino acids therefrom, and CDR-H3 comprising the amino acid sequence of SEQ ID NO: 52 or a sequence differing in 1 or 2 amino acids therefrom: and
   (ii) a VL comprising complementarity-determining regions: CDR-L1 comprising the amino acid sequence of SEQ ID NO: 54 or a sequence differing in 1 or 2 amino acids therefrom, CDR-L2 comprising the amino acid sequence of SEQ ID NO: 55 or a sequence differing in 1 or 2 amino acids therefrom, and CDR-L3 comprising the amino acid sequence of SEQ ID NO: 56 or a sequence differing in 1 or 2 amino acids therefrom:
(h) (i) a VH comprising complementarity-determining regions: CDR-H1 comprising the amino acid sequence of SEQ ID NO: 58 or a sequence differing in 1 or 2 amino acids therefrom, CDR-H2 comprising the amino acid sequence of SEQ ID NO: 59 or a sequence differing in 1 or 2 amino acids therefrom, and CDR-H3 comprising the amino acid sequence of SEQ ID NO: 60 or a sequence differing in 1 or 2 amino acids therefrom; and
   (ii) a VL comprising complementarity-determining regions: CDR-L1 comprising the amino acid sequence of SEQ ID NO: 62 or a sequence differing in 1 or 2 amino acids therefrom, CDR-L2 comprising the amino acid sequence of SEQ ID NO: 63 or a sequence differing in 1 or 2 amino acids therefrom, and CDR-L3 comprising the amino acid sequence of SEQ ID NO: 64 or a sequence differing in 1 or 2 amino acids therefrom:
(i) (i) a VH comprising complementarity-determining regions: CDR-H1 comprising the amino acid sequence of SEQ ID NO: 66 or a sequence differing in 1 or 2 amino acids therefrom, CDR-H2 comprising the amino acid sequence of SEQ ID NO: 67 or a sequence differing in 1 or 2 amino acids therefrom, and CDR-H3 comprising the amino acid sequence of SEQ ID NO: 68 or a sequence differing in 1 or 2 amino acids therefrom; and
   (ii) a VL comprising complementarity-determining regions: CDR-L1 comprising the amino acid sequence of SEQ ID NO: 70 or a sequence differing in 1 or 2 amino acids therefrom, CDR-L2 comprising the amino acid sequence of SEQ ID NO: 71 or a sequence differing in 1 or 2 amino acids therefrom, and CDR-L3 comprising the amino acid sequence of SEQ ID NO: 72 or a sequence differing in 1 or 2 amino acids therefrom:
(j) (i) a VH comprising complementarity-determining regions: CDR-H1 comprising the amino acid sequence of SEQ ID NO: 74 or a sequence differing in 1 or 2 amino acids therefrom, CDR-H2 comprising the amino acid sequence of SEQ ID NO: 75 or a sequence differing in 1 or 2 amino acids therefrom, and CDR-H3 comprising the amino acid sequence of SEQ ID NO: 76 or a sequence differing in 1 or 2 amino acids therefrom: and
   (ii) a VL comprising complementarity-determining regions: CDR-L1 comprising the amino acid sequence of SEQ ID NO: 78 or a sequence differing in 1 or 2 amino acids therefrom, CDR-L2 comprising the amino acid sequence of SEQ ID NO: 79 or a sequence differing in 1 or 2 amino acids therefrom, and CDR-L3 comprising the amino acid sequence of SEQ ID NO: 80 or a sequence differing in 1 or 2 amino acids therefrom:
(k) (i) a VH comprising complementarity-determining regions: CDR-H1 comprising the amino acid sequence of SEQ ID NO: 82 or a sequence differing in 1 or 2 amino acids therefrom, CDR-H2 comprising the amino acid sequence of SEQ ID NO: 83 or a sequence differing in 1 or 2 amino acids therefrom, and CDR-H3 comprising the amino acid sequence of SEQ ID NO: 84 or a sequence differing in 1 or 2 amino acids therefrom: and
   (ii) a VL comprising complementarity-determining regions: CDR-L1 comprising the amino acid sequence of SEQ ID NO: 86 or a sequence differing in 1 or 2 amino acids therefrom, CDR-L2 comprising the amino acid sequence of SEQ ID NO: 87 or a sequence differing in 1 or 2 amino acids therefrom, and CDR-L3 comprising the amino acid sequence of SEQ ID NO: 88 or a sequence differing in 1 or 2 amino acids therefrom:
(l) (i) a VH comprising complementarity-determining regions: CDR-H1 comprising the amino acid sequence of SEQ ID NO: 90 or a sequence differing in 1 or 2 amino acids therefrom, CDR-H2 comprising the amino acid sequence of SEQ ID NO: 91 or a sequence differing in 1 or 2 amino acids therefrom, and CDR-H3 comprising the amino acid sequence of SEQ ID NO: 92 or a sequence differing in 1 or 2 amino acids therefrom: and
   (ii) a VL comprising complementarity-determining regions: CDR-L1 comprising the amino acid sequence of SEQ ID NO: 94 or a sequence differing in 1 or 2 amino acids therefrom, CDR-L2 comprising the amino acid sequence of SEQ ID NO: 95 or a sequence differing in 1 or 2 amino acids therefrom, and CDR-L3 comprising the amino acid sequence of SEQ ID NO: 96 or a sequence differing in 1 or 2 amino acids therefrom:
(m) (i) a VH comprising complementarity-determining regions: CDR-H1 comprising the amino acid sequence of SEQ ID NO: 98 or a sequence differing in 1 or 2 amino acids therefrom, CDR-H2 comprising the amino acid sequence of SEQ ID NO: 99 or a sequence differing in 1 or 2 amino acids therefrom, and CDR-H3 comprising the amino acid sequence of SEQ ID NO: 100 or a sequence differing in 1 or 2 amino acids therefrom: and
   (ii) a VL comprising complementarity-determining regions: CDR-L1 comprising the amino acid sequence of SEQ ID NO: 102 or a sequence differing in 1 or 2 amino acids therefrom, CDR-L2 comprising the amino acid sequence of SEQ ID NO: 103 or a sequence differing in 1 or 2 amino acids therefrom, and CDR-L3 comprising the amino acid sequence of SEQ ID NO: 104 or a sequence differing in 1 or 2 amino acids therefrom: or
(n) (i) a VH comprising complementarity-determining regions: CDR-H1 comprising the amino acid sequence of SEQ ID NO: 106 or a sequence differing in 1 or 2 amino acids therefrom, CDR-H2 comprising the amino acid sequence of SEQ ID NO: 107 or a sequence differing in 1 or 2 amino acids therefrom, and CDR-H3 comprising the amino acid sequence of SEQ ID NO: 108 or a sequence differing in 1 or 2 amino acids therefrom: and
   (ii) a VL comprising complementarity-determining regions: CDR-L1 comprising the amino acid sequence of SEQ ID NO: 110 or a sequence differing in 1 or 2 amino acids therefrom, CDR-L2 comprising the amino acid sequence of SEQ ID NO: 111 or a sequence differing in 1 or 2 amino acids therefrom, and CDR-L3 comprising the amino acid sequence of SEQ ID NO: 112 or a sequence differing in 1 or 2 amino acids therefrom.

In some embodiments, the CDR-H1, CDR-H2, CDR-H3, CDR-L1, CDR-L2, and CDR-L3 comprise amino acid sequences that collectively differ by no more than two amino acid residues from the sequences of:
(a) SEQ ID NO: 2, SEQ ID NO: 3, SEQ ID NO: 4, SEQ ID NO: 6, SEQ ID NO: 7, and SEQ ID NO: 8, respectively:
(b) SEQ ID NO: 10, SEQ ID NO: 11, SEQ ID NO: 12, SEQ ID NO: 14, SEQ ID NO: 15, and SEQ ID NO: 16, respectively:
(c) SEQ ID NO: 18, SEQ ID NO: 19, SEQ ID NO: 20, SEQ ID NO: 22, SEQ ID NO: 23, SEQ ID NO: 24, respectively:
(d) SEQ ID NO: 26, SEQ ID NO: 27, SEQ ID NO: 28, SEQ ID NO: 30, SEQ ID NO: 31, SEQ ID NO: 32, respectively:
(e) SEQ ID NO: 34, SEQ ID NO: 35, SEQ ID NO: 36, SEQ ID NO: 38, SEQ ID NO: 39, SEQ ID NO: 40, respectively:
(f) SEQ ID NO: 42, SEQ ID NO: 43, SEQ ID NO: 44, SEQ ID NO: 46, SEQ ID NO: 47, SEQ ID NO: 48, respectively:
(g) SEQ ID NO: 50, SEQ ID NO: 51, SEQ ID NO: 52, SEQ ID NO: 54, SEQ ID NO: 55, SEQ ID NO: 56, respectively:
(h) SEQ ID NO: 58, SEQ ID NO: 59, SEQ ID NO: 60, SEQ ID NO: 62, SEQ ID NO: 63, SEQ ID NO: 64, respectively:
(i) SEQ ID NO: 66, SEQ ID NO: 67, SEQ ID NO: 68, SEQ ID NO: 70, SEQ ID NO: 71, SEQ ID NO: 72, respectively:
(j) SEQ ID NO: 74, SEQ ID NO: 75, SEQ ID NO: 76, SEQ ID NO: 78, SEQ ID NO: 79, SEQ ID NO: 80, respectively:
(k) SEQ ID NO: 82, SEQ ID NO: 83, SEQ ID NO: 84, SEQ ID NO: 86, SEQ ID NO: 87, SEQ ID NO: 88, respectively:
(l) SEQ ID NO: 90, SEQ ID NO: 91, SEQ ID NO: 92, SEQ ID NO: 94, SEQ ID NO: 95, SEQ ID NO: 96, respectively:
(m) SEQ ID NO: 98, SEQ ID NO: 99, SEQ ID NO: 100, SEQ ID NO: 102, SEQ ID NO: 103, SEQ ID NO: 104, respectively: or
(n) SEQ ID NO: 106, SEQ ID NO: 107, SEQ ID NO: 108, SEQ ID NO: 110, SEQ ID NO: 111, SEQ ID NO: 112, respectively.

In some embodiments, the antibody or antigen binding fragment thereof comprises:
(a) (i) a VH comprising complementarity-determining regions: CDR-H1 comprising the amino acid sequence of SEQ ID NO: 2, CDR-H2 comprising the amino acid sequence of SEQ ID NO: 3, and CDR-H3 comprising the amino acid sequence of SEQ ID NO: 4; and
   (ii) a VL comprising complementarity-determining regions: CDR-L1 comprising the amino acid sequence of SEQ ID NO: 6, CDR-L2 comprising the amino acid sequence of SEQ ID NO: 7, and CDR-L3 comprising the amino acid sequence of SEQ ID NO: 8;
(b) (i) a VH comprising complementarity-determining regions: CDR-H1 comprising the amino acid sequence of SEQ ID NO: 10, CDR-H2 comprising the amino acid sequence of SEQ ID NO: 11, and CDR-H3 comprising the amino acid sequence of SEQ ID NO: 12; and
   (ii) a VL comprising complementarity-determining regions: CDR-L1 comprising the amino acid sequence of SEQ ID NO: 14, CDR-L2 comprising the amino acid sequence of SEQ ID NO: 15, and CDR-L3 comprising the amino acid sequence of SEQ ID NO: 16;
(c) (i) a VH comprising complementarity-determining regions: CDR-H1 comprising the amino acid sequence of SEQ ID NO: 18, CDR-H2 comprising the amino acid sequence of SEQ ID NO: 19, and CDR-H3 comprising the amino acid sequence of SEQ ID NO: 20; and
   (ii) a VL comprising complementarity-determining regions: CDR-L1 comprising the amino acid sequence of SEQ ID NO: 22, CDR-L2 comprising the amino acid sequence of SEQ ID NO: 23, and CDR-L3 comprising the amino acid sequence of SEQ ID NO: 24;
(d) (i) a VH comprising complementarity-determining regions: CDR-H1 comprising the amino acid sequence of SEQ ID NO: 26, CDR-H2 comprising the amino acid sequence of SEQ ID NO: 27, and CDR-H3 comprising the amino acid sequence of SEQ ID NO: 28; and
   (ii) a VL comprising complementarity-determining regions: CDR-L1 comprising the amino acid sequence of SEQ ID NO: 30. CDR-L2 comprising the amino acid sequence of SEQ ID NO: 31. and CDR-L3 comprising the amino acid sequence of SEQ ID NO: 32;
(e) (i) a VH comprising complementarity-determining regions: CDR-H1 comprising the amino acid sequence of SEQ ID NO: 34. CDR-H2 comprising the amino acid sequence of SEQ ID NO: 35. and CDR-H3 comprising the amino acid sequence of SEQ ID NO: 36; and
   (ii) a VL comprising complementarity-determining regions: CDR-L1 comprising the amino acid sequence of SEQ ID NO: 38. CDR-L2 comprising the amino acid sequence of SEQ ID NO: 39. and CDR-L3 comprising the amino acid sequence of SEQ ID NO: 40;
(f) (i) a VH comprising complementarity-determining regions: CDR-H1 comprising the amino acid sequence of SEQ ID NO: 42. CDR-H2 comprising the amino acid sequence of SEQ ID NO: 43. and CDR-H3 comprising the amino acid sequence of SEQ ID NO: 44: and
   (ii) a VL comprising complementarity-determining regions: CDR-L1 comprising the amino acid sequence of SEQ ID NO: 46. CDR-L2 comprising the amino acid sequence of SEQ ID NO: 47. and CDR-L3 comprising the amino acid sequence of SEQ ID NO: 48;
(g) (i) a VH comprising complementarity-determining regions: CDR-H1 comprising the amino acid sequence of SEQ ID NO: 50. CDR-H2 comprising the amino acid sequence of SEQ ID NO: 51. and CDR-H3 comprising the amino acid sequence of SEQ ID NO: 52; and
   (ii) a VL comprising complementarity-determining regions: CDR-L1 comprising the amino acid sequence of SEQ ID NO: 54. CDR-L2 comprising the amino acid sequence of SEQ ID NO: 55. and CDR-L3 comprising the amino acid sequence of SEQ ID NO: 56;
(h) (i) a VH comprising complementarity-determining regions: CDR-H1 comprising the amino acid sequence of SEQ ID NO: 58. CDR-H2 comprising the amino acid sequence of SEQ ID NO: 59. and CDR-H3 comprising the amino acid sequence of SEQ ID NO: 60; and
   (ii) a VL comprising complementarity-determining regions: CDR-L1 comprising the amino acid sequence of SEQ ID NO: 62. CDR-L2 comprising the amino acid sequence of SEQ ID NO: 63. and CDR-L3 comprising the amino acid sequence of SEQ ID NO: 64;
(i) (i) a VH comprising complementarity-determining regions: CDR-H1 comprising the amino acid sequence of SEQ ID NO: 66. CDR-H2 comprising the amino acid sequence of SEQ ID NO: 67. and CDR-H3 comprising the amino acid sequence of SEQ ID NO: 68; and
   (ii) a VL comprising complementarity-determining regions: CDR-L1 comprising the amino acid sequence of SEQ ID NO: 70. CDR-L2 comprising the amino acid sequence of SEQ ID NO: 71. and CDR-L3 comprising the amino acid sequence of SEQ ID NO: 72;
(j) (i) a VH comprising complementarity-determining regions: CDR-H1 comprising the amino acid sequence of SEQ ID NO: 74, CDR-H2 comprising the amino acid sequence of SEQ ID NO: 75, and CDR-H3 comprising the amino acid sequence of SEQ ID NO: 76; and
   (ii) a VL comprising complementarity-determining regions: CDR-L1 comprising the amino acid sequence of SEQ ID NO: 78, CDR-L2 comprising the amino acid sequence of SEQ ID NO: 79, and CDR-L3 comprising the amino acid sequence of SEQ ID NO: 80;
(k) (i) a VH comprising complementarity-determining regions: CDR-H1 comprising the amino acid sequence of SEQ ID NO: 82. CDR-H2 comprising the amino acid sequence of SEQ ID NO: 83, and CDR-H3 comprising the amino acid sequence of SEQ ID NO: 84; and
   (ii) a VL comprising complementarity-determining regions: CDR-L1 comprising the amino acid sequence of SEQ ID NO: 86, CDR-L2 comprising the amino acid sequence of SEQ ID NO: 87, and CDR-L3 comprising the amino acid sequence of SEQ ID NO: 88;
(l) (i) a VH comprising complementarity-determining regions: CDR-H1 comprising the amino acid sequence of SEQ ID NO: 90, CDR-H2 comprising the amino acid sequence of SEQ ID NO: 91, and CDR-H3 comprising the amino acid sequence of SEQ ID NO: 92; and
   (ii) a VL comprising complementarity-determining regions: CDR-L1 comprising the amino acid sequence of SEQ ID NO: 94, CDR-L2 comprising the amino acid sequence of SEQ ID NO: 95, and CDR-L3 comprising the amino acid sequence of SEQ ID NO: 96;
(m) (i) a VH comprising complementarity-determining regions: CDR-H1 comprising the amino acid sequence of SEQ ID NO: 98, CDR-H2 comprising the amino acid sequence of SEQ ID NO: 99, and CDR-H3 comprising the amino acid sequence of SEQ ID NO: 100; and
   (ii) a VL comprising complementarity-determining regions: CDR-L1 comprising the amino acid sequence of SEQ ID NO: 102. CDR-L2 comprising the amino acid sequence of SEQ ID NO: 103, and CDR-L3 comprising the amino acid sequence of SEQ ID NO: 104; or
(n) (i) a VH comprising complementarity-determining regions: CDR-H1 comprising the amino acid sequence of SEQ ID NO: 106, CDR-H2 comprising the amino acid sequence of SEQ ID NO: 107, and CDR-H3 comprising the amino acid sequence of SEQ ID NO: 108; and
   (ii) a VL comprising complementarity-determining regions: CDR-L1 comprising the amino acid sequence of SEQ ID NO: 110, CDR-L2 comprising the amino acid sequence of SEQ ID NO: 111, and CDR-L3 comprising the amino acid sequence of SEQ ID NO: 112.

In some embodiments, the antibody or antigen binding fragment thereof comprises: (i) a VH comprising complementarity-determining regions: CDR-H1 comprising the amino acid sequence of SEQ ID NO: 2: CDR-H2 comprising the amino acid sequence of SEQ ID NO: 3; and CDR-H3 comprising the amino acid sequence of SEQ ID NO: 4: and (ii) a VL comprising complementarity-determining regions: CDR-L1 comprising the amino acid sequence of SEQ ID NO: 6: CDR-L2 comprising the amino acid sequence of SEQ ID NO: 7;and CDR-L3 comprising the amino acid sequence of SEQ ID NO: 8.

In some embodiments, the antibody or antigen binding fragment thereof comprises: a VH comprising an amino acid sequence that is at least 85% identical to that of SEQ ID NO: 1: and a VL comprising an amino acid sequence that is at least 85% identical to that of SEQ ID NO: 5.

In some embodiments, the antibody or antigen binding fragment thereof comprises: a VH comprising the amino acid sequence of SEQ ID NO: 1: and a VL comprising the amino acid sequence of SEQ ID NO: 5.

In some embodiments, the antibody or antigen binding fragment thereof comprises: (i) a VH comprising complementarity-determining regions: CDR-H1 comprising the amino acid sequence of SEQ ID NO: 82: CDR-H2 comprising the amino acid sequence of SEQ ID NO: 83; and CDR-H3 comprising the amino acid sequence of SEQ ID NO: 84; and (ii) a VL comprising complementarity-determining regions: CDR-L1 comprising the amino acid sequence of SEQ ID NO: 86; CDR-L2 comprising the amino acid sequence of SEQ ID NO: 87; and CDR-L3 comprising the amino acid sequence of SEQ ID NO: 88.

In some embodiments, the antibody or antigen binding fragment thereof comprises: a VH comprising an amino acid sequence that is at least 85% identical to that of SEQ ID NO: 81; and a VL comprising an amino acid sequence that is at least 85% identical to that of SEQ ID NO: 85.

In some embodiments, the antibody or antigen binding fragment thereof comprises: the VH comprises the amino acid sequence of SEQ ID NO: 81: and the VL comprises the amino acid sequence of SEQ ID NO: 85.

In some embodiments, the antibody or antigen binding fragment thereof comprises:
(a) a VH comprising an amino acid sequence at least 90%, 95%, 96%, 97%, 98%, or 99% identical to SEQ ID NO: 1 and a VL comprising an amino acid sequence at least 90%, 95%, 96%, 97%, 98%, or 99% identical to SEQ ID NO: 5;
(b) a VH comprising an amino acid sequence at least 90%, 95%, 96%, 97%, 98%, or 99% identical to SEQ ID NO: 9 and a VL comprising an amino acid sequence at least 90%, 95%, 96%, 97%, 98%, or 99% identical to SEQ ID NO: 13;
(c) a VH comprising an amino acid sequence at least 90%, 95%, 96%, 97%, 98%, or 99% identical to SEQ ID NO: 17 and a VL comprising an amino acid sequence at least 90%, 95%, 96%, 97%, 98%, or 99% identical to SEQ ID NO: 21;
(d) a VH comprising an amino acid sequence at least 90%, 95%, 96%, 97%, 98%, or 99% identical to SEQ ID NO: 25 and a VL comprising an amino acid sequence at least 90%, 95%, 96%, 97%, 98%, or 99% identical to SEQ ID NO: 29;
(e) a VH comprising an amino acid sequence at least 90%, 95%, 96%, 97%, 98%, or 99% identical to SEQ ID NO: 33 and a VL comprising an amino acid sequence at least 90%, 95%, 96%, 97%, 98%, or 99% identical to SEQ ID NO: 37;
(f) a VH comprising an amino acid sequence at least 90%, 95%, 96%, 97%, 98%, or 99% identical to SEQ ID NO: 41 and a VL comprising an amino acid sequence at least 90%, 95%, 96%, 97%, 98%, or 99% identical to SEQ ID NO: 45;
(g) a VH comprising an amino acid sequence at least 90%, 95%, 96%, 97%, 98%, or 99% identical to SEQ ID NO: 49 and a VL comprising an amino acid sequence at least 90%, 95%, 96%, 97%, 98%, or 99% identical to SEQ ID NO: 53;
(h) a VH comprising an amino acid sequence at least 90%, 95%, 96%, 97%, 98%, or 99% identical to SEQ ID NO: 57 and a VL comprising an amino acid sequence at least 90%, 95%, 96%, 97%, 98%, or 99% identical to SEQ ID NO: 61;
(i) a VH comprising an amino acid sequence at least 90%, 95%, 96%, 97%, 98%, or 99% identical to SEQ ID NO: 65 and a VL comprising an amino acid sequence at least 90%, 95%, 96%, 97%, 98%, or 99% identical to SEQ ID NO: 69;
(j) a VH comprising an amino acid sequence at least 90%, 95%, 96%, 97%, 98%, or 99% identical to SEQ ID NO: 73 and a VL comprising an amino acid sequence at least 90%, 95%, 96%, 97%, 98%, or 99% identical to SEQ ID NO: 77;
(k) a VH comprising an amino acid sequence at least 90%, 95%, 96%, 97%, 98%, or 99% identical to SEQ ID NO: 81 and a VL comprising an amino acid sequence at least 90%, 95%, 96%, 97%, 98%, or 99% identical to SEQ ID NO: 85;
(l) a VH comprising an amino acid sequence at least 90%, 95%, 96%, 97%, 98%, or 99% identical to SEQ ID NO: 89 and a VL comprising an amino acid sequence at least 90%, 95%, 96%, 97%, 98%, or 99% identical to SEQ ID NO: 93;
(m) a VH comprising an amino acid sequence at least 90%, 95%, 96%, 97%, 98%, or 99% identical to SEQ ID NO: 97 and a VL comprising an amino acid sequence at least 90%, 95%, 96%, 97%, 98%, or 99% identical to SEQ ID NO: 101; or
(n) a VH comprising an amino acid sequence at least 90%, 95%, 96%, 97%, 98%, or 99% identical to SEQ ID NO: 105 and a VL comprising an amino acid sequence at least 90%, 95%, 96%, 97%, 98%, or 99% identical to SEQ ID NO: 109.

In some embodiments, the antibody or antigen binding fragment thereof comprises:
(a) a VH comprising an amino acid sequence of SEQ ID NO: 1 and a VL comprising an amino acid sequence of SEQ ID NO: 5;
(b) a VH comprising an amino acid sequence of SEQ ID NO: 9 and a VL comprising an amino acid sequence of SEQ ID NO: 13;
(c) a VH comprising an amino acid sequence of SEQ ID NO: 17 and a VL comprising an amino acid sequence of SEQ ID NO: 21;
(d) a VH comprising an amino acid sequence of SEQ ID NO: 25 and a VL comprising an amino acid sequence of SEQ ID NO: 29;
(e) a VH comprising an amino acid sequence of SEQ ID NO: 33 and a VL comprising an amino acid sequence of SEQ ID NO: 37;
(f) a VH comprising an amino acid sequence of SEQ ID NO: 41 and a VL comprising an amino acid sequence of SEQ ID NO: 45;
(g) a VH comprising an amino acid sequence of SEQ ID NO: 49 and a VL comprising an amino acid sequence of SEQ ID NO: 53;
(h) a VH comprising an amino acid sequence of SEQ ID NO: 57 and a VL comprising an amino acid sequence of SEQ ID NO: 61;
(i) a VH comprising an amino acid sequence of SEQ ID NO: 65 and a VL comprising an amino acid sequence of SEQ ID NO: 69;
(j) a VH comprising an amino acid sequence of SEQ ID NO: 73 and a VL comprising an amino acid sequence of SEQ ID NO: 77;
(k) a VH comprising an amino acid sequence of SEQ ID NO: 81 and a VL comprising an amino acid sequence of SEQ ID NO: 85;
(l) a VH comprising an amino acid sequence of SEQ ID NO: 89 and a VL comprising an amino acid sequence of SEQ ID NO: 93;
(m) a VH comprising an amino acid sequence of SEQ ID NO: 97 and a VL comprising an amino acid sequence of SEQ ID NO: 101; or
(n) a VH comprising an amino acid sequence of SEQ ID NO: 105 and a VL comprising an amino acid sequence of SEQ ID NO: 109.

In some embodiments, the antibody or antigen binding fragment thereof is capable of binding to human Claudin 18.2 with an EC₅₀ value of at most about 10 nM or lower, at most about 8 nM or lower, at most about 6 nM or lower, at most about 5 nM or lower, at most about 4 nM or lower, or at most about 3 nM or lower. In some embodiments, the EC₅₀ value is determined by a flow cytometry assay as described in Example 4. In some embodiments, the antibody or antigen binding fragment thereof is capable of binding to mouse Claudin 18.2. In some embodiments, the antibody or antigen binding fragment thereof is not capable of significantly binding to Claudin 18.1.

In some embodiments, the antibody or antigen binding fragment thereof is capable of being internalized after contacting with cells expressing Claudin 18.2.

In some embodiments, the antibody or antigen binding fragment thereof is a human antibody. In some embodiments, the antibody or antigen binding fragment thereof is a Fab, a F(ab)₂, a Fab′, a single-chain Fv(scFv), an Fv fragment, a Fd fragment, or a diabody. In some embodiments, the antibody or antigen binding fragment thereof is an scFv.

In some embodiments, the antibody or antigen binding fragment thereof comprises an antibody heavy chain constant region. In some embodiments, the the heavy chain constant region is a human IgG heavy chain constant region. In some embodiments, the antibody heavy chain constant region is a human IgG4 heavy chain constant region. In some embodiments, the human IgG4 heavy chain constant region comprises a S228P mutation.

In some embodiments, the antibody or antigen binding fragment thereof is conjugated to a therapeutic agent. In some embodiments, the antibody or antigen binding fragment thereof is conjugated to a label.

In another aspect, provided are isolated nucleic acids encoding an antibody or antigen binding fragment thereof of described herein. In some embodiments, the nucleic acid is comprised in an expression vector. In some embodiments, the isolated nucleic acid or the expression vector is comprised in an host cell.

In another aspect, provided are composition comprising an antibody or antigen binding fragment thereof described herein and a pharmaceutically acceptable carrier. In some embodiments, the composition further comprising a therapeutic agent.

In another aspect, provided are methods of treating cancer in a subject, the methods comprising administering to the subject an effective amount of the antibody or antigen binding fragment thereof or antibody conjugate described herein.

In another aspect, provided are methods of detecting the presence of Claudin 18.2, or a fragment thereof, in a sample, the methods comprising contacting the sample with an antibody or antigen binding fragment described herein and detecting the presence of a complex between the antibody or antigen binding fragment and Claudin 18.2, wherein detection of the complex indicates the presence of Claudin 18.2.

### BRIEF DESCRIPTION OF THE DRAWINGS

FIGS. 1A and 1B depict Claudin 18.2 expression on MiaPaCa-2 cells analyzed by flow cytometry after stable transfection and clone isolation (FIG. 1A) and after two weeks of cultivation (FIG. 1B).

FIGS. 2A-2N depict the binding of antibody clones D03 (FIG. 2A), E07 (FIG. 2B), H03 (FIG. 2C), D10 (FIG. 2D), E12 (FIG. 2E), A02 (FIG. 2F), F02 (FIG. 2G), D11 (FIG. 2H), H09 (FIG. 21), B11 (FIG. 2J), E08 (FIG. 2K), H07 (FIG. 2L), D01 (FIG. 2M), and claudiximab (FIG. 2N) to hCLDN18.2-expressing MiaPaCa-2 cells.

FIGS. 3A-3F depict binding of claudiximab (FIG. 3A) and antibody clones D03 (FIG. 3B), D10 (FIG. 3C), H03 (FIG. 3D), and H07 (FIG. 3E), and H09 (FIG. 3F) to hCLDN18.2-expressing MiaPaCa-2 cells and CLDN 18.2 endogenously expressing PATU8988S cells.

FIG. 4 shows the internalization of antibody clones H09, H03, D03, D10, and H07 and claudiximab after contacting hCLDN18.2-expressing MiaPaCa-2 cells.

### DETAILED DESCRIPTION

### Definitions

As used herein, the terms “about,” “approximately,” and “comparable to,” when used herein in reference to a value, refer to a value that is similar to the referenced value in the context of that referenced value. In general, those skilled in the art, familiar with the context, will appreciate the relevant degree of variance encompassed by “about,” “approximately,” and “comparable to” in that context. For example, in some embodiments, the terms “about,” “approximately,” and “comparable to” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

As used herein, the terms “antagonistic,” “neutralizing” or “blocking,” when used in reference to an antibody or antigen-binding fragment thereof, is intended to refer to an antibody or fragment thereof whose binding to its target results in inhibition of at least some of the biological activity of the target.

As used herein, “antibody” refers to a polypeptide whose amino acid sequence includes immunoglobulins and fragments thereof which specifically bind to a designated antigen, or fragments thereof. Antibodies in accordance with the present invention may be of any type (e.g., IgA, IgD, IgE, IgG, or IgM) or subtype (e.g., IgA1, IgA2, IgG1, IgG2, IgG3, or lgG4). Those of ordinary skill in the art will appreciate that a characteristic sequence or portion of an antibody may include amino acids found in one or more regions of an antibody (e.g., variable region, hypervariable region, constant region, heavy chain, light chain, and combinations thereof). Moreover, those of ordinary skill in the art will appreciate that a characteristic sequence or portion of an antibody may include one or more polypeptide chains, and may include sequence elements found in the same polypeptide chain or in different polypeptide chains.

“Antibody-dependent cell-mediated cytotoxicity” or “ADCC” refers to a form of cytotoxicity in which secreted Ig bound onto Fc receptors (FcRs) present on certain cytotoxic cells (e.g. Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model.

An “antigen-binding fragment” of an antibody, or “antibody fragment” comprises a portion of an intact antibody, which portion is still capable of antigen binding. In some embodiments, the antibody has a function in addition to that of antigen-binding, and an antigen-binding fragment retains that function. Typically, an antigen-binding fragment comprises the variable region of the antibody. Papain digestion of antibodies produce two identical antigen-binding fragments, called “Fab” fragments, and a residual “Fc” fragment, a designation reflecting the ability to crystallize readily. The Fab fragment consists of an entire light chain along with the variable region domain of the heavy chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and that is still capable of cross-linking antigen. Fab′ fragments differ from Fab fragments by having a few additional residues at the carboxy terminus of the C_{H}1 domain, including one or more cysteines from the antibody hinge region. Fab′-SH designates an Fab′ in which the cysteine residue(s) of the constant domains bear a free thiol group. F(ab′)₂ antibody fragments originally were produced as pairs of Fab′ fragments having hinge cysteines between them. Other chemical couplings of antibody fragments are also known.

A “complementarity determining region” (abbreviated “CDR”) is a region of hypervariability interspersed within regions that are more conserved, termed “framework regions” (abbreviated “FR”). In some embodiments, the sequences of the framework regions are identical to the framework regions in human germline sequences. In some embodiments, the sequences of the framework regions are modified with respect to the human germline sequence.

As used herein, the phrase “complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. Assays to assess complement activation are known in the art.

As used herein, the expression “control sequences” refers to DNA sequences necessary or advantageous for the expression of an operably linked coding sequence in a particular host organism. Control sequences that are typically suitable for prokaryotes, for example, include a promoter, optionally an operator sequence, and a ribosome binding site. Eukaryotic cells are known to utilize promoters, polyadenylation signals, and enhancers.

As used herein, antibody “effector functions” refer to those biological activities attributable to the Fc region (a native sequence Fc region or amino acid sequence variant Fc region) of an antibody, and which typically vary with the antibody isotype. Examples of antibody effector functions include, but are not limited to, C1q binding and complement dependent cytotoxicity, Fc receptor binding: antibody-dependent cell-mediated cytotoxicity (ADCC): phagocytosis, down regulation of cell surface receptors (e.g. B cell receptor), and B cell activation.

As used herein, the term “epitope” is an antigenic determinant that interacts with a specific antigen binding site in the variable region of an antibody molecule, known as the paratope, and which is comprised of the six complementary-determining regions of the antibody. A single antigen may have more than one epitope. Epitopes may be conformational or linear. A conformational epitope is comprised of spatially juxtaposed amino acids from different segments of a linear polypeptide chain. A linear epitope is comprised of adjacent amino acid residues in a polypeptide chain.

An “Fc” fragment comprises the carboxy-terminal portions of both heavy chains held together by disulfides. The effector functions of antibodies are determined by sequences in the Fc region, the region which is also recognized by Fc receptors (FcR) found on certain types of cells.

As used herein, an “isolated” nucleic acid molecule is a nucleic acid molecule that is identified and separated from at least one contaminant molecule with which it is ordinarily associated in the natural source of the antibody nucleic acid. An isolated nucleic acid molecule is other than in the form or setting in which it is found in nature. Isolated nucleic acid molecules therefore are distinguished from the nucleic acid molecule as they exists in natural cells.

As used herein, the term “monoclonal antibody” refers to an antibody obtained from a population of substantially homogeneous antibodies, e.g., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, monoclonal antibodies may be made by a hybridoma method, or may be made by recombinant DNA methods (see, e.g., U.S. Pat. No. 4,816,567). “Monoclonal antibodies” may also be isolated from phage antibody libraries.

As used herein, a nucleic acid is “operably linked” when it is placed into a functional relationship with another nucleic acid sequence. For example, DNA for a presequence or secretory leader is “operably linked” to DNA for a polypeptide if it is expressed as a preprotein that participates in the secretion of the polypeptide: a promoter or enhancer is “operably linked” to a coding sequence if it affects the transcription of the sequence: or a ribosome binding site is “operably linked” to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking may be accomplished, e.g., by ligation at convenient restriction sites. If such sites do not exist, synthetic oligonucleotide adaptors or linkers may be used in accordance with conventional practice.

As used herein, “polypeptide” refers to a string of at least two amino acids attached to one another by a peptide bond. In some embodiments, a polypeptide may include at least 3-5 amino acids, each of which is attached to others by way of at least one peptide bond. Those of ordinary skill in the art will appreciate that polypeptides can include one or more “non-natural” amino acids or other entities that nonetheless are capable of integrating into a polypeptide chain. In some embodiments, a polypeptide may be glycosylated, e.g., a polypeptide may contain one or more covalently linked sugar moieties. In some embodiments, a single “polypeptide” (e.g., an antibody polypeptide) may comprise two or more individual polypeptide chains, which may in some cases be linked to one another, for example by one or more disulfide bonds or other means.

As used herein, the phrase “reference level” generally refers to a level considered “normal” for comparison purposes, e.g., a level of an appropriate control. For example, in the context of tumor growth inhibition or reduction, a “reference level” may refer to the level of tumor growth expected in a subject not receiving a therapeutic agent of interest (e.g., the level of tumor growth in a subject before the subject is administered a therapeutic agent of interest, or the level of tumor growth in another subject who is not receiving a therapeutic agent of interest), or in a subject receiving a treatment (e.g., the current standard of care) other than the therapeutic agent of interest. A reference level may be determined contemporaneously or may be predetermined, e.g., known or deduced from past observations.

As used herein, the phrases “therapeutically effective amount” and “effective amount” are used interchangeably and refer to an amount effective, at dosages and for periods of time necessary, to achieve a desired therapeutic result. A therapeutically effective amount may vary according to factors such as the type of disease (e.g., cancer), disease state, age, sex, and/or weight of the individual, and the ability of an immunoconjugate (or pharmaceutical composition thereof) to elicit a desired response in the individual. An effective amount may also be an amount for which any toxic or detrimental effects of the immunoconjugate or pharmaceutical composition thereof are outweighed by therapeutically beneficial effects.

As used herein, to “treat” a condition or “treatment” of the condition (e.g., the conditions described herein such as cancer) is an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e., not worsening) state of disease, disorder, or condition; preventing spread of disease, disorder, or condition (e.g., of a primary cancer and/or of a secondary metastases); delay or slowing the progress of the disease, disorder, or condition; amelioration or palliation of the disease, disorder, or condition; and remission (whether partial or total), whether detectable or undetectable. “Palliating” a disease, disorder, or condition means that the extent and/or undesirable clinical manifestations of the disease, disorder, or condition are lessened and/or time course of the progression is slowed or lengthened, as compared to the extent or time course in the absence of treatment.

### Antibodies and Antigen-Binding Fragments Thereof

In one aspect, provided are antibodies and antigen-binding fragments thereof that are capable of binding to Claudin 18.2. In some embodiments, the antibodies or antigen-binding fragments are monoclonal antibodies. In some embodiments, the antibodies or antigen-binding fragments are human antbodies.

In some embodiments, provided are Claudin 18.2-binding fragments. For example, the fragments may be, e.g., an scFv, an Fab, an scFab (single-chain Fab), an F(ab)₂, a Fab′, a single-chain Fv (scFv), an Fv fragment, a Fd fragment, or a diabody

As used herein, the term “scFv” is used in accordance with its common usage in the art to refer to a single chain in which the V_{H} domain and the V_{L} domain from an antibody are joined, typically via a linker.

As used herein, the term “Fab fragment” is used in accordance with its common usage in the art. Fab fragments typically comprise an entire light chain (V_{L} and C_{L}1 domains), the variable region domain of the heavy chain (V_{H}), and the first constant domain of one heavy chain (C_{H}1).

In some embodiments, provided are immunoconjugates comprising an antibody or antigen-binding fragment as disclosed herein that are labeled and/or conjugated to a cytotoxic agent such as a toxin or a radioactive isotope.

### Exemplary Antibodies

In some embodiments, provided antibodies or antigen-binding fragments comprise a heavy chain variable domain and a light chain variable domain, wherein the heavy chain variable domain comprises a CDR-H1, CDR-H2, and CDR-H3, and the light chain variable domain comprises a CDR-L1, CDR-L2, and CDR-L3, wherein the CDR-H1, CDR-H2, CDR-H3, CDR-L1, CDR-L2, and CDR-L3, are those of an antibody described in Table 1 below. In some embodiments, provided are antibodies or antigen-binding fragments that are variants of the antibodies shown in Table 1, in that such antibodies or antigen-binding fragments have CDR sequences that differ by no more than two amino acid residues (e.g., two or one amino acid residue(s)) per CDR from the CDR sequences of an antibody described in Table 1. In some embodiments, provided are antibodies or antigen-binding fragments that are variants of the antibodies shown in Table 1, in that such antibodies or antigen-binding fragments have a set of six CDRs whose sequences collectively differ by no more than two amino acid residues (e.g., two or one amino acid residues) from the CDRs of an antibody described in Table 1.

In some embodiments, provided antibodies or antigen-binding fragments comprise a heavy chain variable domain and a light chain variable domain which comprise heavy chain variable domain and light chain variable sequences of an antibody described in Table 1. In some embodiments, provided are antibodies or antigen-binding fragments that are variants of the antibodies shown in Table 1, in that such antibodies or antigen-binding fragments have (1) a heavy chain domain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of the heavy chain variable domain of an antibody described in Table 1: and (2) a light chain domain comprising an amino acid sequence that is at least 85%, at least 87.5%, at least 90%, at least 91%, at least 92%, at least 93%, at least 94%, at least 95%, at least 96%, at least 97%, at least 98%, or at least 99% identical to the amino acid sequence of a light chain variable domain of the same antibody described in Table 1.

TABLE 1
Exemplary heavy chain variable domain, light chain variable domain, and
complementarity-determining region sequences of Claudin 18.2 antibodies

| CLDN18 | | |
|---|---|---|
| Antibody | Heavy chain variable domain | Light chain variable domain |
| YU574-D03 | EVQLLESGGGLVQPGGSLRLSCAASG | DIVMTQSPSSVSASVGDRVTITCRAS |
| | FTFSSYAMSWVRQAPGKGLEWVSAIS | QGISSWLAWYQQKPGKAPKLLIYAAS |
| | GSGGSTYYADSVKGRFTISRDNSKNT | SLQSGVPSRFSGSGSGTDFTLTISSL |
| | LYLQMNSLRAEDTAVYYCAGRQVAWL | QPEDFATYYCQQANSFPLTFGGGTKV |
| | GNWGQGTLVTVSS | DIK |
| | (SEQ ID NO: 1) | (SEQ ID NO: 5) |
| | CDR-H1: SYAMS | CDR-L1: RASQGISSWLA |
| | (SEQ ID NO: 2) | (SEQ ID NO: 6) |
| | CDR-H2: AISGSGGSTYYADSVKG | CDR-L2: AASSLQS |
| | (SEQ ID NO: 3) | (SEQ ID NO: 7) |
| | CDR-H3: RQVAWLGN | CDR-L3: QQANSFPLT |
| | (SEQ ID NO: 4) | (SEQ ID NO: 8) |
| YU574-D11 | QVQLVQSGGGLVQPGGSLRLSCAASG | QSVLTQPPSVSGAPGQRVTISCTGSS |
| | FTFSSYAMSWVRQAPGKGLEWVSAIS | SNIGAGYDVHWYQQLPGTAPKLLIYG |
| | GSGGSTYYADSVKGRFTISRDNSKNT | NSNRPSGVPDRFSGSKSGTSASLAIT |
| | LYLQMNSLRAEDTAVYYCAGGANGFD | GLQAEDEADYYCQSYDSSLSGLVFGG |
| | PWGQGTLVTVSS | GTKLTVL |
| | (SEQ ID NO: 9) | (SEQ ID NO: 13) |
| | CDR-H1: SYAMS | CDR-L1: TGSSSNIGAGYDVH |
| | (SEQ ID NO: 10) | (SEQ ID NO: 14) |
| | CDR-H2: AISGSGGSTYYADSVKG | CDR-L2: GNSNRPS |
| | (SEQ ID NO: 11) | (SEQ ID NO: 15) |
| | CDR-H3: GANGFDP | CDR-L3: QSYDSSLSGLV |
| | (SEQ ID NO: 12) | (SEQ ID NO: 16) |
| YU574-E07 | QVQLQESGGGVVQPGRSLRLSCAASG | QSVLTQPPSASGAPGQRVTISCTGSS |
| | FTFSSYGMHWVRQAPGKGLEWVAVIS | SNIGAGYDVHWYQQLPGTAPKLLIYG |
| | YDGSNKYYADSVKGRFTISRDNSKNT | NSNRPSGVPDRFSGSKSGTSASLAIT |
| | LYLQMNSLRAEDTAVYYCAKDQRGGL | GLQAEDEADYYCQSYDSSLSGWVFGG |
| | YGMDVWGQGTLVTVSS | GTKLTVL |
| | (SEQ ID NO: 17) | (SEQ ID NO: 21) |
| | CDR-H1: SYGMH | CDR-L1: TGSSSNIGAGYDVH |
| | (SEQ ID NO: 18) | (SEQ ID NO: 22) |
| | CDR-H2: VISYDGSNKYYADSVKG | CDR-L2: GNSNRPS |
| | (SEQ ID NO: 19) | (SEQ ID NO: 23) |
| | CDR-H3: DQRGGLYGMDV | CDR-L3: QSYDSSLSGWV |
| | (SEQ ID NO: 20) | (SEQ ID NO: 24) |
| YU574-H09 | QVQLVQSGAEVKKPGASVKVSCKASG | SYVLTQPPSASGTPGQRVTISCSGSS |
| | YTFTSYYMHWVRQAPGQGLEWMGIIN | SNIGSNYVYWYQQLPGTAPKLLIYSN |
| | PSGGSTSYAQKFQGRVTMTRDTSTST | NQRPSGVPDRFSGSKSGTSASLAISG |
| | VYMELSSLRSEDTAVYYCARGGSGFD | LRSEDEADYYCAAWDDSLSGWVFGGG |
| | YWGQGTLVTVSS | TKLTVL |
| | (SEQ ID NO: 25) | (SEQ ID NO: 29) |
| | CDR-H1: SYYMH | CDR-L1: SGSSSNIGSNYVY |
| | (SEQ ID NO: 26) | (SEQ ID NO: 30) |
| | CDR-H2: IINPSGGSTSYAQKFQG | CDR-L2: SNNQRPS |
| | (SEQ ID NO: 27) | (SEQ ID NO: 31) |
| | CDR-H3: GGSGFDY | CDR-L3: AAWDDSLSGWV |
| | (SEQ ID NO: 28) | (SEQ ID NO: 32) |
| YU575-H03 | EVQLVQSGGGLVQPGGSLRLSCAASG | QSVLTQPPSVSGAPGQRVTISCTGSS |
| | FTFSSYAMSWVRQAPGKGLEWVSAIS | SNIGAGYDVHWYQQLPGTAPKLLIYG |
| | GSGGSTYYADSVKGRFTISRDNSKNT | NSNRPSGVPDRFSGSKSGTSASLAIT |
| | LYLQMNSLRAEDTAVYYCAIPGDYGS | GLQAEDEADYYCQSYDSSLSGYVFGT |
| | SDPWGQGTLVTVSS | GTKLTVL |
| | (SEQ ID NO: 33) | (SEQ ID NO: 37) |
| | CDR-H1: SYAMS | CDR-L1: TGSSSNIGAGYDVH |
| | (SEQ ID NO: 34) | (SEQ ID NO: 38) |
| | CDR-H2: AISGSGGSTYYADSVKG | CDR-L2: GNSNRPS |
| | (SEQ ID NO: 35) | (SEQ ID NO: 39) |
| | CDR-H3: PGDYGSSDP | CDR-L3: QSYDSSLSGYV |
| | (SEQ ID NO: 36) | (SEQ ID NO: 40) |
| YU577-B01 | EVQLVESGGGVVQPGRSLRLSCAASG | DIQLTQSPSSLSASVGDRVTITCRAS |
| | FTFSSYGMHWVRQAPGKGLEWVAVIS | QGISNYLAWFQQKPGKAPKSLIYAAS |
| | YDGSNKYYADSVKGRFTISRDNSKNT | SLQSGVPSKFSGSGSGTDFTLTISSL |
| | LYLQMNSLRAEDTAVYYCAKDVTYYY | QPEDFATYYCQQYNSYPYSFGQGTKV |
| | DSSGSPDFDYWGQGTLVTVSS | EIK |
| | (SEQ ID NO: 41) | (SEQ ID NO: 45) |
| | CDR-H1: SYGMH | CDR-L1: RASQGISNYLA |
| | (SEQ ID NO: 42) | (SEQ ID NO: 46) |
| | CDR-H2: VISYDGSNKYYADSVKG | CDR-L2: AASSLQS |
| | (SEQ ID NO: 43) | (SEQ ID NO: 47) |
| | CDR-H3: DVTYYYDSSGSPDFDY | CDR-L3: QQYNSYPYS |
| | (SEQ ID NO: 44) | (SEQ ID NO: 48) |
| YU577-B11 | EVQLVESGGGLVQPGGSLRLSCAASG | DIQMTQSPSSVSASVGDRVTITCRAS |
| | FTFSSYAMSWVRQAPGKGLEWVSAIS | QGISSWLAWYQQKPGKAPKLLIYAAS |
| | GSGGSTYYADSVKGRFTISRDNSKNT | SLQSGVPSRFSGSGSGTDFTLTISSL |
| | LYLQMNSLRAEDTAVYYCAGYTGGAF | QPEDFATYYCQQANSFPLTFGGGTKL |
| | DIWGQGTMVTVSS | EIK |
| | (SEQ ID NO: 49) | (SEQ ID NO: 53) |
| | CDR-H1: SYAMS | CDR-L1: RASQGISSWLA |
| | (SEQ ID NO: 50) | (SEQ ID NO: 54) |
| | CDR-H2: AISGSGGSTYYADSVKG | CDR-L2: AASSLQS |
| | (SEQ ID NO: 51) | (SEQ ID NO: 55) |
| | CDR-H3: YTGGAFDI | CDR-L3: QQANSFPLT |
| | (SEQ ID NO: 52) | (SEQ ID NO: 56) |
| YU577-D10 | QVQLVQSGGGLVQPGGSLRLSCAASG | DIQMTQSPSSLSASVGDRVTITCQAS |
| | FTFSSYAMSWVRQAPGKGLEWVSAIS | QDISNYLNWYQQKPGKAPKLLIYDAS |
| | GSGGSTYYADSVKGRFTISRDNSKNT | NLETGVPSRFSGSGSGTDFTFTISSL |
| | LYLQMNSLRAEDTAVYYCAKPLRFLG | QPEDIATYYCQQYDNLPLTFGGGTKL |
| | DYGGWGQGTLVTVSS | EIK |
| | (SEQ ID NO: 57) | (SEQ ID NO: 61) |
| | CDR-H1: SYAMS | CDR-L1: QASQDISNYLN |
| | (SEQ ID NO: 58) | (SEQ ID NO: 62) |
| | CDR-H2: AISGSGGSTYYADSVKG | CDR-L2: DASNLET |
| | (SEQ ID NO: 59) | (SEQ ID NO: 63) |
| | CDR-H3: PLRFLGDYGG | CDR-L3: QQYDNLPLT |
| | (SEQ ID NO: 60) | (SEQ ID NO: 64) |
| YU577-E08 | QVQLVQSGAEVKKPGASVKVSCKASG | QSVLTQPPSVSAAPGQKVTISCSGSS |
| | YTFTSYYMHWVRQAPGQGLEWMGIIN | SNIGNNYVSWYQQLPGTAPKLLIYEN |
| | PSGGSTSYAQKFQGRVTMTRDTSTST | NKRPSGIPDRFSGSKSGTSATLGITG |
| | VYMELSSLRSEDTAVYYCATRQFDYW | LQTGDEADYYCGTWDSSLSAAVFGGG |
| | GQGTLVTVSS | TKVTVL |
| | (SEQ ID NO: 65) | (SEQ ID NO: 69) |
| | CDR-H1: SYYMH | CDR-L1: SGSSSNIGNNYVS |
| | (SEQ ID NO: 66) | (SEQ ID NO: 70) |
| | CDR-H2: IINPSGGSTSYAQKFQG | CDR-L2: ENNKRPS |
| | (SEQ ID NO: 67) | (SEQ ID NO: 71) |
| | CDR-H3: RQFDY | CDR-L3: GTWDSSLSAAV |
| | (SEQ ID NO: 68) | (SEQ ID NO: 72) |
| YU577-E12 | QVQLVESGGGLVQPGGSLRLSCAASG | DIVMTQSPSFLSASAGDRVTITCRAS |
| | FTFSSYAMSWVRQAPGKGLEWVSAIS | QGISSYLAWYQQKPGKGPKLLIYAAS |
| | GSGGSTYYADSVKGRFTISRDNSKNT | TLQSGVPSRFSGSGSGTEFTLTISSL |
| | LYLQMNSLRAEDTAVYYCAKDLGVYY | QPEDFATYYCQQVNSYPLTFGPGTKV |
| | FDYWGQGTLVTVSS | DIK |
| | (SEQ ID NO: 73) | (SEQ ID NO: 77) |
| | CDR-H1: SYAMS | CDR-L1: RASQGISSYLA |
| | (SEQ ID NO: 74) | (SEQ ID NO: 78) |
| | CDR-H2: AISGSGGSTYYADSVKG | CDR-L2: AASTLOS |
| | (SEQ ID NO: 75) | (SEQ ID NO: 79) |
| | CDR-H3: DLGVYYFDY | CDR-L3: QQVNSYPLT |
| | (SEQ ID NO: 76) | (SEQ ID NO: 80) |
| YU577-H07 | EVQLLESGGGLVQPGGSLRLSCAASG | DIQMTQSPSSVSASVGDRVTITCRAS |
| | FTFSSYAMSWVRQAPGKGLEWVSAIS | QGISSWLAWYQQKPGKAPKLLIYAAS |
| | GSGGSTYYADSVKGRFTISRDNSKNT | SLQSGVPSRFSGSGSGTDFTLTISSL |
| | LYLQMNSLRAEDTAVYYCAKEPFYGM | QPEDFATYYCQQANSFPLTFGGGTKL |
| | DVWGQGTLVTVSS | EIK |
| | (SEQ ID NO: 81) | (SEQ ID NO: 85) |
| | CDR-H1: SYAMS | CDR-L1: RASQGISSWLA |
| | (SEQ ID NO: 82) | (SEQ ID NO: 86) |
| | CDR-H2: AISGSGGSTYYADSVKG | CDR-L2: AASSLQS |
| | (SEQ ID NO: 83) | (SEQ ID NO: 87) |
| | CDR-H3: EPFYGMDV | CDR-L3: QQANSFPLT |
| | (SEQ ID NO: 84) | (SEQ ID NO: 88) |
| YU578-A02 | EVQLLESGGGLVQPGGSLRLSCAASG | DIVMTQSPSTLSASVGDRVTITCRAS |
| | FTFSSYAMSWVRQAPGKGLEWVSAIS | QSISSWLAWYQQKPGKAPKLLIYKAS |
| | GSGGSTYYADSVKGRFTISRDNSKNT | SLESGVPSRFSGSGSGTEFTLTISSL |
| | LYLQMNSLRAEDTAVYYCAKPPGSYY | QPDDFATYYCQQYNSYPLTFGGGTKV |
| | VWDYWGQGTLVTVSS | EIK |
| | (SEQ ID NO: 89) | (SEQ ID NO: 93) |
| | CDR-H1: SYAMS | CDR-L1: RASQSISSWLA |
| | (SEQ ID NO: 90) | (SEQ ID NO: 94) |
| | CDR-H2: AISGSGGSTYYADSVKG | CDR-L2: KASSLES |
| | (SEQ ID NO: 91) | (SEQ ID NO: 95) |
| | CDR-H3: PPGSYYVWDY | CDR-L3: QQYNSYPLT |
| | (SEQ ID NO: 92) | (SEQ ID NO: 96) |
| YU578-D01 | EVQLVQSGGGLVQPGGSLRLSCAASG | DIQLTQSPSSLSASVGDRVTITCRAS |
| | FTFSSYAMSWVRQAPGKGLEWVSAIS | QGISNYLAWFQQKPGKAPKSLIYAAS |
| | GSGGSTYYADSVKGRFTISRDNSKNT | SLQSGVPSKFSGSGSGTDFTLTISSL |
| | LYLQMNSLRAEDTAVYYCAKTAGSYY | QPEDFATYYCQQYNSYPLTFGGGTKV |
| | ILDYWGQGTLVTVSS | DIK |
| | (SEQ ID NO: 97) | (SEQ ID NO: 101) |
| | CDR-H1: SYAMS | CDR-L1: RASQGISNYLA |
| | (SEQ ID NO: 98) | (SEQ ID NO: 102) |
| | CDR-H2: AISGSGGSTYYADSVKG | CDR-L2: AASSLQS |
| | (SEQ ID NO: 98) | (SEQ ID NO: 103) |
| | CDR-H3: TAGSYYILDY | CDR-L3: QQYNSYPLT |
| | (SEQ ID NO: 100) | (SEQ ID NO: 104) |
| YU578-F02 | QVQLVESGGGLVQPGGSLRLSCAASG | DIVMTQSPSFLSASVGDRVTITCRAS |
| | FTFSSYAMSWVRQAPGKGLEWVSAIS | QGISSYLAWYQQKPGKAPKLLIYAAS |
| | GSGGSTYYADSVKGRFTISRDNSKNT | TLQSGVPSRFSGSGSGTEFTLTISSL |
| | LYLQMNSLRAEDTAVYYCAKPLTAGY | QPEDFATYYCQQLNSYPITFGQGTKL |
| | YYGMVAFDIWGQGTMVTVSS | EIK |
| | (SEQ ID NO: 105) | (SEQ ID NO: 109) |
| | CDR-H1: SYAMS | CDR-L1: RASQGISSYLA |
| | (SEQ ID NO: 106) | (SEQ ID NO: 110) |
| | CDR-H2: AISGSGGSTYYADSVKG | CDR-L2: AASTLQS |
| | (SEQ ID NO: 107) | (SEQ ID NO: 111) |
| | CDR-H3: PLTAGYYYGMVAFDI | CDR-L3: QQLNSYPIT |
| | (SEQ ID NO: 112) | (SEQ ID NO: 108) |

### Antibody Fragments

In certain embodiments, provided are antibody fragments, rather than whole antibodies.

Various techniques have been developed for the production of antibody fragments. Traditionally, these fragments were derived via proteolytic digestion of intact antibodies However, these fragments can now be produced directly by recombinant host cells. Fab, Fv and ScFv antibody fragments can all be expressed in and secreted from E. coli, thus allowing the production of large amounts of these fragments. Antibody fragments can be isolated from, e.g., antibody phage libraries. Alternatively or additionally, Fab′-SH fragments can be directly recovered from E. coli and chemically coupled to form F(ab′)₂ fragments. According to another approach, F(ab′)₂ fragments can be isolated directly from recombinant host cell culture. Fab and F(ab′)₂ fragment with increased in vivo half-life comprising a salvage receptor binding epitope residues are described, e.g., in U.S. Pat. No. 5,869,046. Other techniques for the production of antibody fragments will be apparent to the skilled practitioner.

In some embodiments, the antigen-binding fragment is a single chain Fv fragment (scFv). See, e.g., WO 93/16185; U.S. Pat. No. 5,571,894; and U.S. Pat. No. 5,587,458. Fv and scFv are species with intact combining sites that are devoid of constant regions: thus, these fragments may be suitable for reduced nonspecific binding during in vivo use. scFv fusion proteins may be constructed to yield fusion of an effector protein at either the amino or the carboxy terminus of an scFv. An antigen-binding antibody fragment may also be a “linear antibody”, e.g., as described in U.S. Pat. No. 5,641,870.

### Amino Acid Sequence Modifications

Amino acid sequence modification(s) of the antibodies or antigen-binding fragments disclosed herein are contemplated. For example, it may be desirable to improve the binding affinity and/or other biological properties of the antibodies or antigen-binding fragments. Amino acid sequence variants can be prepared, e.g., by introducing appropriate nucleotide changes into a nucleic acid sequence encoding the antibody or antigen-binding fragment, or by peptide synthesis. Such modifications can include, for example, deletions from, and/or insertions into and/or substitutions of, residues within the amino acid sequences of the antibody or antigen-binding fragment. Any combination of deletion, insertion, and substitution can be made, provided that the antibody or antigen-binding fragment has the desired characteristics. In some embodiments, amino acid changes are introduced to alter post-translational processes, such as changing the number or position of glycosylation sites.

A useful method for identification of certain residues or regions that are preferred locations for mutagenesis is called “alanine scanning mutagenesis.” In this method, a residue or group of target residues are identified (e.g., charged residues such as arg, asp, his, lys, and glu) and replaced by a neutral or negatively charged amino acid (most preferably alanine or polyalanine) to affect the interaction of the amino acids with antigen. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis may be conducted at the target codon or region, and the expressed variants may be screened for a desired activity.

Examples of amino acid sequence insertions include, but are not limited to, amino-and/or carboxyl-terminal fusions ranging in length from one residue to polypeptides containing a hundred or more residues, as well as intrasequence insertions of single or multiple amino acid residues. An example of a terminal insertion includes, but are not limited to, N-terminal methionyl residues.

In some embodiments, the antibody or antigen-binding fragment is fused at one terminus to another polypeptide, e.g., a cytotoxic polypeptide, an enzyme, or a polypeptide which increases the serum half-life of the antibody or antigen-binding fragment.

Another type of variant is an amino acid substitution variant. These variants have at least one amino acid residue in the antibody molecule or antigen-binding fragment molecule replaced by a different residue. Sites of greatest interest for substitutional mutagenesis are typically the hypervariable regions, but framework region alterations are also contemplated. Examples of conservative substitutions are shown in Table 2 under the heading of “preferred substitutions.” More substantial changes, under the heading “exemplary substitutions” in Table 2, or as further described below in reference to amino acid classes, may be introduced and the resulting antibodies or antigen-binding fragments screened.

TABLE 2
Exemplary Amino acid substitutions

| Original | | Preferred |
|---|---|---|
| Residue | Exemplary Substitutions | Substitutions |
| Ala (A) | Val, Leu, Ile | Val |
| Arg (R) | Lys, Gln, Asn | Lys |
| Asn (N) | Gln, His, Asp, Lys, Arg | Gln |
| Asp (D) | Glu. Asn | Glu |
| Cys (C) | Ser, Ala | Ser |
| Gln (Q) | Asn, Glu | Asn |
| Glu (E) | Asp, Gln | Asp |
| Gly (G) | Ala | Ala |
| His (H) | Asn, Gln, Lys, Arg | Arg |
| Ile (I) | Leu, Val, Met, Ala, Phe, | Leu |
| | Norleucine | |
| Leu (L) | Norleucine, Ile, Val, Met, | Ile |
| | Ala, Phe | |
| Lys (K) | Arg, Gln, Asn | Arg |
| Met (M) | Leu Phe, Ile | Leu |
| Phe (F) | Leu, Val, Ile, Ala, Tyr | Tyr |
| Pro (P) | Ala | Ala |
| Ser (S) | Thr | Thr |
| Thr (T) | Ser | Ser |
| Trp (W) | Tyr, Phe | Tyr |
| Tyr (Y) | Trp, Phe, Thr, Ser | Phe |
| Val (V) | Ile, Leu, Met, Phe, Ala, | Leu |
| | Norleucine | |

Substantial modifications in the biological properties of the antibody may be accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are typically divided into groups based on common side-chain properties:
(1) hydrophobic: Norleucine, Met, Ala, Val, Leu, Ile;
(2) neutral hydrophilic: Cys, Ser, Thr;
(3) acidic: Asp, Glu;
(4) basic: Asn, Gln, His, Lys, Arg;
(5) residues that influence chain orientation: Gly, Pro; and
(6) aromatic: Trp, Tyr, Phe.

Non-conservative substitutions can entail exchanging a member of one of these classes for another class.

Additionally or alternatively, cysteine residues not involved in maintaining the proper conformation of the antibody or antigen-binding fragment may be substituted, generally with serine, to improve the oxidative stability of the molecule and prevent aberrant crosslinking. Conversely, cysteine bond(s) may be added to the antibody to improve its stability (particularly where the antibody is an antibody fragment such as an Fv fragment).

In some embodiments, a substitutional variant comprises a substitution within one or more hypervariable region residues of a parent antibody (e.g. a human antibody). Generally, the resulting variant(s) having improved biological properties relative to the parent antibody from which they are generated are selected for further development.

A method for generating such substitutional variants involves affinity maturation using phage display. In an example of such a method, several hypervariable region sites (e.g., 6-7 sites) are mutated to generate all possible amino substitutions at each site. Antibody variants thus generated are displayed in a monovalent fashion, e.g., from filamentous phage particles as fusions to the gene III product of M13 packaged within each particle. The phage-displayed variants are then screened for their biological activity (e.g., binding affinity).

To identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody or antigen-binding fragment and the antigen. Such contact residues and neighboring residues are candidates for substitution according to the techniques elaborated herein. Once such variants are generated, the panel of variants is subjected to screening, and antibodies with superior properties in one or more relevant assays may be selected for further development.

In some embodiments, the original glycosylation pattern of a parent antibody is altered. Such alteration(s) may comprise deleting one or more carbohydrate moieties found in the antibody, and/or adding one or more glycosylation sites that are not present in the antibody.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are recognition sequences for enzymatic attachment of the carbohydrate moiety to an asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody may be accomplished by altering the antibody or antigen-bindng fragment's amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Nucleic acid molecules encoding amino acid sequence variants of antibodies or antigen-binding fragments may be prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody or antigen-binding fragment thereof.

In some embodiments, antibodies or antigen-binding fragments are modified with respect to effector function, e.g., so as to enhance antigen-dependent cell-mediated cyotoxicity (ADCC) and/or complement dependent cytotoxicity (CDC) of the antibody. Such modification(s) may be achieved, e.g., by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). Homodimeric antibodies with enhanced anti-tumor activity may also be prepared using heterobifunctional cross-linkers. Alternatively or additionally, an antibody can be engineered which has dual Fc regions and may thereby have enhanced complement mediated lysis and ADCC capabilities.

In some embodiments, a modification that increases the serum half life of the antibody or antigen-binding fragment is used. For example, a salvage receptor binding epitope can be incorporated into an antibody (especially an antibody fragment) as described, e.g., in U.S. Pat. No. 5,739,277. As used herein, the term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

### Vectors, Host Cells and Recombinant Methods

Also provided are isolated nucleic acids encoding antibodies and antigen-binding fragments, vectors and host cells comprising the nucleic acid, and recombinant techniques for the production of the antibody.

For recombinant production of the antibody or antigen-binding fragment, a nucleic acid encoding the antibody or antigen-binding fragment may be isolated and inserted into a replicable vector for further cloning (amplification of the DNA) or for expression. DNA encoding the monoclonal antibody can be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

### Pharmaceutical Compositions

In certain embodiments, provided immunoconjugates are incorporated together with one or more pharmaceutically acceptable carriers into a pharmaceutical composition suitable for administration to a subject. As used herein, “pharmaceutically acceptable carrier” refers to any of a variety of solvents, dispersion media, coatings, antibacterial and antifungal agents, isotonic and absorption delaying agents, and the like that are physiologically compatible. Examples of pharmaceutically acceptable carriers include, but are not limited to, water, saline, phosphate buffered saline, dextrose, glycerol, ethanol and the like, as well as combinations thereof.

In some embodiments, pharmaceutical compositions comprise one or more tonicity agents or stabilizers. Non-limiting examples of such tonicity agents or stabilizers include sugars (e.g., sucrose), polyalcohols (e.g., mannitol or sorbitol), and sodium chloride.

In some embodiments, pharmaceutical compositions comprise one or more bulking agents and/or lyoprotectants (e.g., mannitol or glycine), buffers (e.g., phosphate, acetate, or histidine buffers), surfactants (e.g., polysorbates), antioxidants (e.g., methionine), and/or metal ions or chelating agents (e.g., ethylenediaminetetraacetic acid (EDTA)).

In some embodiments, pharmaceutical compositions comprise one or more auxiliary substances such as wetting or emulsifying agents, preservatives (e.g., benzyl alcohol) or buffers, which may enhance the shelf life and/or effectiveness of immunoconjugates disclosed herein.

Pharmaceutical compositions may be provided in any of a variety of forms. These include, for example, liquid, semi-solid and solid dosage forms, such as liquid solutions (e.g., injectable and infusible solutions), dispersions or suspensions, tablets, pills, powders, liposomes and suppositories. Suitability of certain forms may depend on the intended mode of administration and therapeutic application.

In some embodiments, pharmaceutical compositions are in the form of injectable or infusible solutions.

Pharmaceutical compositions are typically sterile and stable under conditions of manufacture, transport, and storage. Pharmaceutical compositions may be formulated as, for example, a solution, microemulsion, dispersion, liposome, or other ordered structure. In some embodiments, a pharmaceutical composition is formulated as a structure particularly suitable for high drug concentration. For example, sterile injectable solutions can be prepared by incorporating a therapeutic agent (e.g., immunoconjugate) in a desired amount in an appropriate solvent with one or a combination of ingredients enumerated herein, optionally followed by sterilization (e.g., filter sterilization). Generally, dispersions may be prepared by incorporating an immunoconjugate into a sterile vehicle that contains a basic dispersion medium and other ingredient(s) such as those additional ingredients mentioned herein. In the case of sterile powders for the preparation of sterile injectable solutions, examples of preparation methods include vacuum drying and freeze-drying to yield a powder of the immunoconjugate and any additional desired ingredient(s), e.g., from a previously sterile-filtered solution thereof.

Proper fluidity of a solution can be maintained, for example, by the use of a coating such as lecithin, by maintaining certain particle sizes (e.g., in the case of dispersions), and/or by using surfactants. Prolonged absorption of injectable compositions can be brought about, e.g., by including in the composition an agent that delays absorption (for example, monostearate salts and/or gelatin).

### Methods of Making Antibodies or Antigen-Binding Fragments

### Monoclonal Antibodies

Monoclonal antibodies may be made using the hy bridoma method first described by, or may be made by recombinant DNA methods (U.S. Pat. No. 4,816,567).

In the hybridoma method, a mouse or other appropriate host animal, such as a hamster, is immunized to elicit lymphocytes that produce or are capable of producing antibodies that will specifically bind to the protein used for immunization. Alternatively or additionally, lymphocytes may be immunized in vitro. After immunization, lymphocytes are isolated and then fused with a myeloma cell line using a suitable fusing agent, such as polyethylene glycol, to form a hybridoma cell.

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Examples of suitable fusion partner include, but are not limited to, myeloma cells are those that fuse efficiently, support stable high-level production of antibody by the selected antibody-producing cells, and are sensitive to a selective medium that selects against the unfused parental cells. Examples of suitable myeloma cell lines include, but are not limited to, are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Rockville, Md. USA. Human myeloma and mouse-human heteromyeloma cell lines also have been described for the production of human monoclonal antibodies.

According to the hy bridoma mehtod, culture medium in which hybridoma cells are growing is then assaved for production of monoclonal antibodies directed against the antigen. For example, the binding specificity of monoclonal antibodies produced by hy bridoma cells may be determined by immunoprecipitation or by an in vitro binding assay, such as radioimmunoassay (RIA) or enzyme-linked immunosorbent assay (ELISA).

The binding affinity of the monoclonal antibody can, for example, be determined by a Scatchard analysis.

Once hybridoma cells that produce antibodies of the desired specificity, affinity, and/or activity are identified, clones may be subcloned, e.g., by limiting dilution procedures and grown by standard methods. Suitable culture media for this purpose include, for example, D-MEM or RPMI-1640 medium. In addition, the hybridoma cells may be grown in vivo as ascites tumors in an animal e.g. by i.p. injection of the cells into mice.

Monoclonal antibodies secreted by the subclones are suitably separated from the culture medium, ascites fluid, or serum by conventional antibody purification procedures such as, for example, affinity chromatography (e.g., using protein A or protein G-Sepharose (R)) or ion-exchange chromatography, hydroxy lapatite chromatography, gel electrophoresis, dialysis, etc.

DNA encoding the monoclonal antibodies can be readily isolated and sequenced using conventional procedures (e.g., by using oligonucleotide probes that are capable of binding specifically to genes encoding the heavy and light chains of murine antibodies). Hybridoma cells can serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells.

In certain embodiments, monoclonal antibodies or antibody fragments are isolated from antibody phage libraries. High affinity (nM range) human antibodies can be produced, e.g., by chain shuffling. Combinatorial infection and in vivo recombination may provide strategies for constructing very large phage libraries. These techniques are viable alternatives to traditional monoclonal antibody hy bridoma techniques for isolation of monoclonal antibodies.

DNA that encodes the antibody may be modified to produce chimeric or fusion antibody polypeptides, for example, by substituting human heavy chain and light chain constant domain (CH and CL) sequences for the homologous murine sequences (see, e.g., U.S. Pat. No. 4,816,567), or by fusing the immunoglobulin coding sequence with all or part of the coding sequence for a non-immunoglobulin polypeptide (heterologous polypeptide).

### Human Antibodies and Phage Display Methodology

Human antibodies can be generated by methods known in the art, including methods described herein. For example, it is possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (JH) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., U.S. Pat. Nos. 5,545,806, 5,569,825, 5,591,669; 5,545,807; and WO 97/17852.

Alternatively, phage display technology can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats. Several sources of V-gene segments can be used for phage display, e.g., from random combinatorial librarues of V genes such as libraries derived from the spleens of immunized mice. A repertoire of V genes from unimmunized human donors can be constructed and antibodies to a diverse array of antigens (including self-antigens) can be isolated essentially following methods described in the art. See, e.g., U.S. Pat. Nos. 5,565,332 and 5,573,905.

Human antibodies may also be generated by in vitro activated B cells (see, e.g. U.S. Pat. Nos. 5,567,610 and 5,229,275).

### Methods of Treatment

Methods of treating cancer disclosed herein generally comprise a step of administering a therapeutically effective amount of an immunoconjugate (or pharmaceutical composition thereof) of the present disclosure to a mammalian subject (e.g., a human subject) in need thereof. In some embodiments, the subject is diagnosed as having cancer.

Therapeutically effective amounts may be administered via a single dose or via multiple doses (e.g., at least two, at least three, at least four, at least five, at least six, at least seven, at least eight, at least nine, or at least ten doses). When administered via multiple doses, any of a variety of suitable therapeutic regimens may be used, including administration at regular intervals (e.g., once every other day, once every three days, once every four days, once every five days, thrice weekly, twice weekly, once a week, once every two weeks, once every three weeks, etc.).

The dosage regimen (e.g., amounts of each therapeutic, relative timing of therapies, etc.) that is effective in methods of treatment may depend on the severity of the disease or condition and the weight and general state of the subject. For example, the therapeutically effective amount of a particular composition comprising a therapeutic agent applied to mammals (e.g., humans) can be determined by the ordinarily-skilled artisan with consideration of individual differences in age, weight, and the condition of the mammal. Therapeutically effective and/or optimal amounts can also be determined empirically by those of skill in the art. In some embodiments, subjects are administered a dose between 0.4 mg/kg every 3 days to 20 mg/kg every 3 days. Immunoconjugates and pharmaceutical compositions thereof may be administered by any of a variety of suitable routes, including, but not limited to, systemic routes such as parenteral (e.g., intravenous or subcutaneous) or enteral routes.

In certain embodiments, the subject is diagnosed with cancer.

In some embodiments, administration results in a measurable improvement in the subject. For example, this improvement may include any or any combination of tumor growth inhibition (TGI), tumor growth reduction, tumor regression, inhibition or reduction of metastases, improved survival, or improvement in any clinical sign indicative of cancer status or progression. Tumor growth may be assessed by measures such as, e.g., estimated or measured tumor volumes. In some embodiments, tumor growth inhibition or reduction is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% (e.g., based on lower tumor volume relative to a reference, such as a reference value representative of a tumor volume in a subject receiving no treatment). In some embodiments, administration results in regression of the tumor, i.e. a decrease in size of a tumor or in extent of cancer in the body relative to the size at the commencement of a therapeutic regimen involving an immunoconjugate. This tumor regression may be partial (i.e., some of the tumor or cancer remains) or complete (e.g., the tumor volume reaches approximately zero and/or the tumor is no longer measurable or detectable).

### EXAMPLES

### Example 1. Generation of Claudin 18.2 Stable Cell Line

The cDNA of human Claudin 18.2 (hCLDN18.2) was synthesized and cloned into a pVBC-PAC vector. The cDNA is driven by an SV40 promoter, and the puromycin acetyltransferase (puromycin resistance gene) is coupled to the promoter bicistronically via a POLIO IRES element.

MiaPaCa-2 cells were transfected with the hCLDN18.2-expressing vector using Lipofectamine 2000 reagent and selected with puromycin. The clonal cell lines were isolated and expanded. Antigen expression was verified by staining the cells with claudiximab (also known as zolbetuximab or IMAB362) and subsequent analysis using flow cytometry.

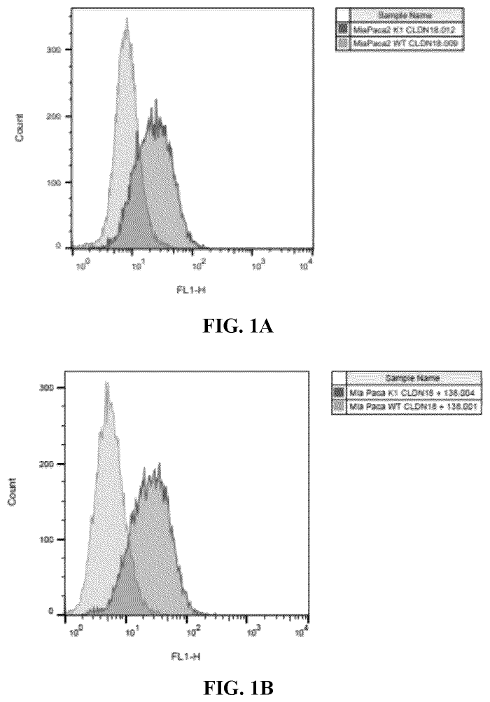

The hCLDN18.2 stable expression cell lines were successfully established. FIG. 1 shows the flow cytometric analysis of hCLDN18.2 expression in an exemplary clone after isolation (FIG. 1A) and after two weeks of cultivation (FIG. 1B).

### Example 2. Antigen Preparation

Full-length human Claudin 18.1 (hCLDN18.1), hCLDN18.2, and mouse Claudin 18.2 (mCLDN18.2) genes were each synthesized and fused with the green fluorescent protein (GFP) gene at the C-terminus. The fusion genes, hCLDN18.1-GFP, hCLDN18.2-GFP, and mCLDN18.2-GFP, were respectively cloned into mammalian expression vectors and transiently transfected into HEK cells.

Additionally, cDNA encoding the first extracellular domain (ECD1) of hCLDN18.1, hCLDN18.2, or mCLDN18.2 was amplified by PCR and fused to mouse Fc (mFc). The fusion genes, hCLDN18.1-ECD1-mFc, hCLDN18.2-ECD1-mFc, and mCLDN18.2-ECDI-mFc, were respectively cloned into a mammalian expression vector and transiently transfected into HEK cells.

The expression of the full-length Claudin-18 on the cell surface was confirmed via the GFP signal. The expression of hCLDN18.2 was further analyzed by staining the transiently transfected HEK cells with a CLDN18.2 antibody.

hCLDN18.1-ECD1-mFc, hCLDN18.2-ECD1-mFc, and mCLDN18.2-ECD1-mFc were expressed, affinity purified, and confirmed using SDS-PAGE.

### Example 3. Identification and Production of Human Antibodies

A naïve human antibody library was used to select CLDN18.2 antibodies and incubated with CLDN18.2 antigen or CLDN18.2-expressing cells for four rounds of biopanning, following the strategy described in Table 3. Monoclonal antibody clones were selected, produced as scFv antibodies, and screened using ELISA (clones identified from Strategy 1 or 2) or flow cytometry (clones identified from Strategy 3 or 4) to identify clones with hCLDN18.2 affinity and no hCLDN18.1 cross-reactivity. 188 such clones were and subsequently sequenced, yielding 68 unique antibodies. The unique antibodies were further screened using flow cytometry to identify clones with mCLDN18.2 affinity, yielding 50 human/mouse cross-reactive antibody clones.

TABLE 3
Antibody selection strategy

| Strategy | Round 1 | Round 2 | Round 3 | Round 4 |
|---|---|---|---|---|
| 1 | hCLDN18.2-ECD1 | hCLDN18.2-ECD1 | hCLDN18.2-ECD1 | hCLDN18.2-ECD1 |
| | protein | protein | protein | protein |
| 2 | hCLDN18.2-ECD1 | mCLDN18.2-ECD1 | hCLDN18.2-ECD1 | mCLDN18.2-ECD1 |
| | protein | protein | protein | protein |
| 3 | hCLDN18.2-expressing | hCLDN18.2-expressing | hCLDN18.2-expressing | hCLDN18.2-expressing |
| | cells | cells | cells | cells |
| 4 | hCLDN18.2-expressing | mCLDN18.2-expressing | hCLDN18.2-expressing | mCLDN18.2-expressing |
| | cells | cells | cells | cells |

Sequences of the 50 human/mouse cross-reactive antibodies were then subjected to in-silico developability assessment, where the 3D models of the antibodies were constructed and used for calculation of key characteristics, including isoelectric point (pI), germinality index (GI), unusual residues, CDR length, hydrophobicity score, charge, amidation site, and glycosylation pattern. The characteristics were compared with therapeutic antibodies and human antibody databases to rank the developability of the putative antibodies.

14 clones were selected for further assessment. Heavy chain variable domain (VH) and light chain variable domain (VL) sequences of each selected antibody were respectively cloned into a human IgG4 S228P expression vector and transiently transfected into HEK cells for antibody production. Expressed antibodies were purified via Protein A affinity chromatography, followed by UV-Vis spectroscopic and SDS-PAGE analyses. Table 4 summarizes the antibody yields.

TABLE 4
Antibody production

| Antibody | Yield [mg/mL] |
|---|---|
| D03 | 0.27 |
| D11 | 0.79 |
| E07 | 0.27 |
| H09 | 0.48 |
| H03 | 0.38 |
| B01 | — |
| B11 | 0.43 |
| D10 | 0.47 |
| E08 | 0.75 |
| E12 | 0.52 |
| H07 | 0.31 |
| A02 | 0.18 |
| D01 | 0.08 |
| F02 | 0.47 |

### Example 4. Binding of Antibodies Toward CLDN18.2 Analyzed by Flow Cytometry

Target binding of CLDN18.2 antibodies identified in Example 3 to CLDN18.2-expressing cells was assessed by flow cytometry.

MiaPaCa-2 cells were stably transfected with hCLDN18.2 as described in Example 1. hCLDN18.2-expressing MiaPaCa-2 cells or wild-type (WT) MiaPaCa-2 cells were incubated with dilution series of the CLDN18.2 antibodies and detected using a fluorescently labeled anti-human IgG antibody. Mean geometric fluorescent signals were plotted and fitted. Claudiximab was tested as a positive control.

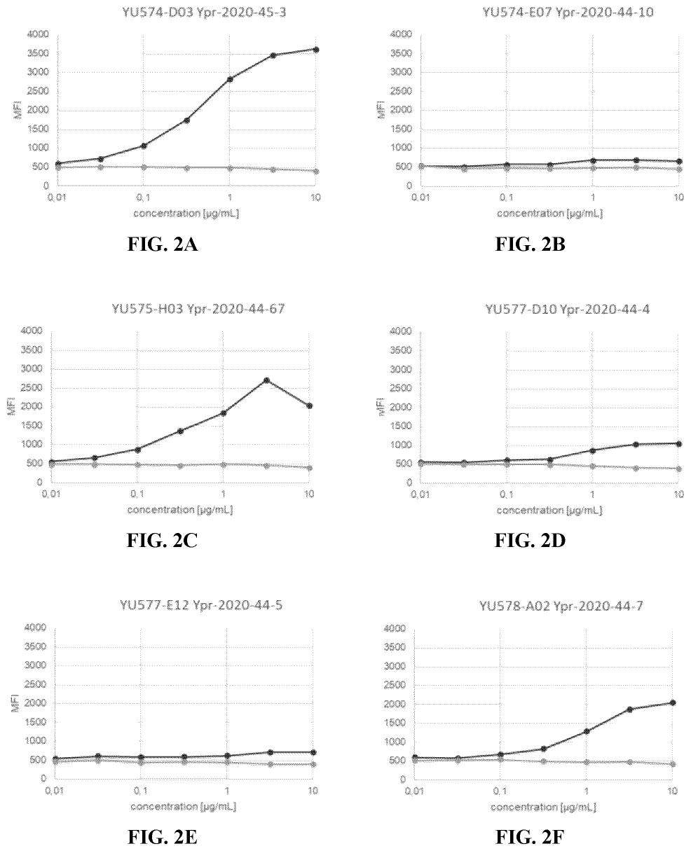

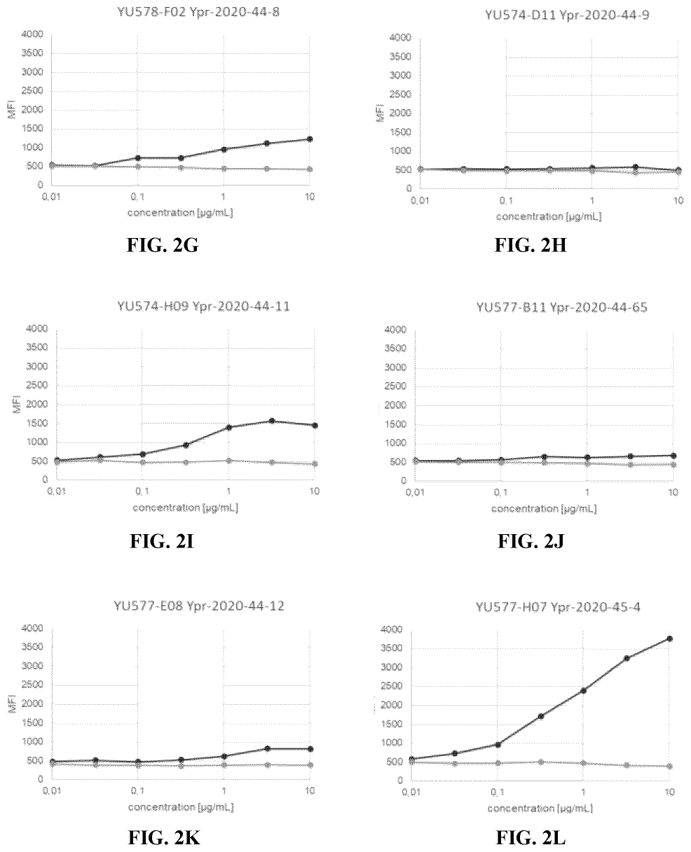

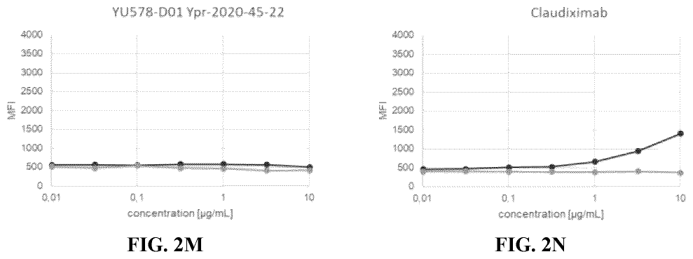

The ability of exemplary CLDN18.2 antibodies to bind hCLDN18.2 is depicted in FIGS. 2A-2N. Binding affinities (EC50s) to hCLDN18.2-expressing cells of the CLDN18.2 antibodies are in the single digit nanomolar range, comparable to claudiximab, which is a previously known CLDN18.2 antibody (see Table 5).

TABLE 5
Binding affinities of the human CLDN18.2
antibodies to hCLDN18.2-expressing cells

| Antibody Clone | EC₅₀ [nM] |
|---|---|
| H09 | 2.54 |
| D03 | 2.88 |
| H03 | 3.48 |
| F02 | 3.88 |
| H07 | 4.21 |
| D10 | 4.93 |
| A02 | 6.36 |
| E08 | 7.20 |
| Claudiximab | 8.63 |

### Example 5. Binding of Antibodies Toward CLDN18.2 Analyzed by Flow Cytometry

An additional flow cytometric analysis was used to analyze the target binding of CLDN18.2 antibodies identified in Example 3 to CLDN18.2-expressing cells.

hCLDN18.2-expressing MiaPaCa2 cells and PATU8988S cells were counted and suspended in 96-well plates at the density of 5×10⁵ cells/well. Claudin 18.2 antibody clones D03, D10, H03, H07, and H09, were added to the respective wells at ˜1.5 μg/mL antibody concentration in FACS and incubated for 30 min at 4° C. The plate was centrifuged and then washed, followed by the addition of R-phycoerythrin (PE)-conjugated goat anti-human Fc antibody to each well and 15 min incubation at 4° C. in the dark. The plate was subsequently washed, and the cell pellets were resuspended in FACS buffer. The fluorescence signals were captured using BD Accuri C6 Plus flow cytometer and analyzed using FlowJo software version 10.2. Claudin 18.2 antibody claudiximab was used as a positive control.

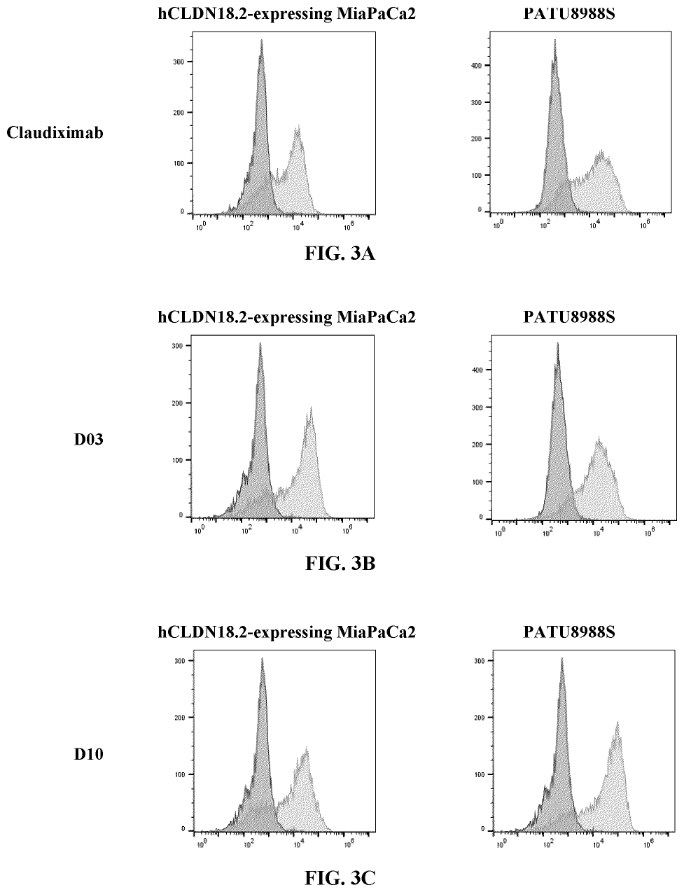

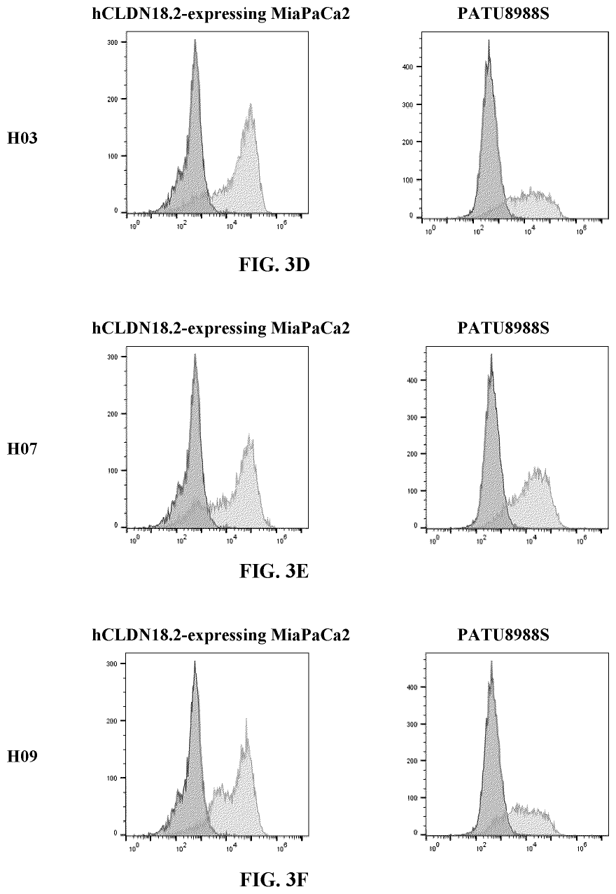

The ability of exemplary CLDN18.2 antibodies to bind hCLDN18.2 is depicted in FIGS. 3A-3F. D03 and H07 antibody clones demonstrated comparable binding ability to claudiximab in both MiaPaCa2 and PATU8988S cells.

### Example 6. Analysis of Antibody Internalization

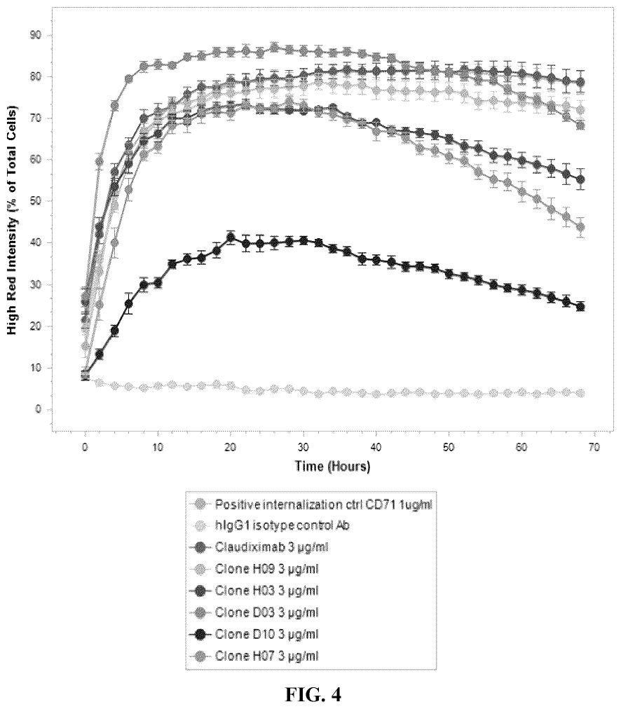

hCLDN18.2-expressing MiaPaCa-2 cells were counted and suspended in 96-well plates at the density of 5000 cells/well. Exemplary Claudin 18.2 antibody clones identified in Example 3 or claudiximab were mixed with FabFluor pH antibody labeling dye and added to the respective wells at ˜3 μg/mL antibody concentration. CD71 antibody was used as a positive control for internalization. The plate was then immediately placed in the Incucyte® Live-Cell Analysis System for live cell imaging. The cell images were captured every 2 hours for 72 hours. Cell-by-cell analysis was carried out, and the red dye intensity uptake (% to total cells) graph was plotted (FIG. 4).

Internalization was observed for all tested clones, D03, D10, H03, H07, and H09,and is comparable amongst different clones as well as to the control antibodies used.

### Example 7. Non-Specific Binding of Antibodies Analyzed by ELISA

An ELISA-based assay was employed to assess the non-specific binding of CLDN18.2 antibodies identified in Example 3 toward DNA, lipopolysaccharide (LPS), lysozyme, and cell lysate.

DNA, LPS, lysozyme, and cell lysate were immobilized onto microtiter plates. Testing antibodies were added to the wells and incubated. Bound antibodies were detected via a fluorescently-labeled substrate. The fluorescence intensity was normalized to the signal of a control antibody (trastuzumab). The normalized signals of each testing antibody binding to DNA, LPS, lysozyme, and cell lysate are shown in Table 6. Higher normalized signal correlates to stronger off-target or non-specific binding. H03, D10, A02, and F02 antibody clones showed similar polyreactivity as the control antibody and lower non-specificity compared to DO2, H09, and H07 antibody clones.

TABLE 6
Non-specific binding of human CLDN18.2 antibodies

| Antibody Clone | DNA | LPS | Lysozyme | Cell lysate | Mean |
|---|---|---|---|---|---|
| D03 | 1 | 1.2 | 1.1 | 1.3 | 1.2 |
| H09 | 1 | 1.1 | 1.3 | 1.9 | 1.3 |
| H03 | 0.9 | 1 | 0.9 | 1 | 1 |
| D10 | 0.9 | 1 | 0.9 | 1 | 0.9 |
| H07 | 1.3 | 1.1 | 1.3 | 1.5 | 1.3 |
| A02 | 1 | 1.1 | 1 | 0.9 | 1 |
| F02 | 0.9 | 1 | 1 | 0.9 | 0.9 |
| Claudiximab | 0.9 | 1.1 | 0.9 | 1.2 | 1 |
| Trastuzumab | 1 | 1 | 1 | 1 | 1 |

### Example 8. Stability Assessment

To assess stability, antibody clones D03, H09, H03, D10, H07, A02, and F02 were incubated for 48 hours at 45° C. or for 24 hours at pH 3.0. Monomeric antibodies were subsequently determined using size exclusion. Trastuzumab was used as a reference antibody. D03, D10, and H07 showed a comparable or lower degree of aggregation as trastuzumab under both heat and pH stress conditions.

TABLE 7
Stability of human CLDN18.2 antibodies

| Antibody | | High MW fraction | Monomeric fraction | Low MW fraction |
|---|---|---|---|---|
| Clone | Treatment | (>180 kDa) [%] | (180-130 kDa) [%] | (<130 kDa) [%] |
| Trastuzumab | — | 12.7 | 87.2 | 0.0 |
| | pH stress | 28.4 | 71.5 | 0.0 |
| | heat stress | 10.8 | 88.5 | 0.6 |
| D03 | — | 14.4 | 85.5 | 0.0 |
| | pH stress | 10.1 | 89.9 | 0.0 |
| | heat stress | 4.7 | 93.4 | 1.8 |
| H09 | — | 19.9 | 80.1 | 0.0 |
| | pH stress | 34.8 | 65.2 | 0.0 |
| | heat stress | 14.8 | 84.6 | 0.6 |
| H03 | — | 19.4 | 80.6 | 0.0 |
| | pH stress | 0.0 | 100.0 | 0.0 |
| | heat stress | 13.0 | 86.5 | 0.5 |
| D10 | — | 13.5 | 86.5 | 0.0 |
| | pH stress | 27.6 | 72.4 | 0.0 |
| | heat stress | 9.0 | 90.4 | 0.5 |
| H07 | — | 14.3 | 85.7 | 0.0 |
| | pH stress | 29.4 | 70.6 | 0.0 |
| | heat stress | 11.7 | 87.9 | 0.3 |
| A02 | — | 15.9 | 84.1 | 0.0 |
| | pH stress | 22.8 | 77.2 | 0.0 |
| | heat stress | 6.0 | 93.4 | 0.5 |
| F02 | — | 16.5 | 0.0 | 83.5 |
| | pH stress | 29.9 | 0.0 | 70.0 |
| | heat stress | 10.0 | 0.0 | 90.0 |

The thermal stability of the antibodies was assessed by their melting temperatures (TM) and aggregation temperatures (TM_{agg}), measured using nanoscale differential scanning fluorimetry (nanoDSF).

TABLE 8
Thermal stability of human CLDN18.2 antibodies

| Antibody Clone | TM1 [° C.] | TM2 [° C.] | TM_{agg} [° C.] |
|---|---|---|---|
| D03 | 73.12 | | 72.61 |
| H09 | 69.74 | 76.86 | 75.53 |
| H03 | 72.44 | | 71.29 |
| D10 | 68.59 | 79.12 | 78.85 |
| H07 | 69.83 | 75.42 | 73.51 |
| A02 | 74.22 | | 73.82 |
| F02 | 67.63 | 75.80 | 74.97 |
| Trastuzumab | 69.45 | 76.09 | 74.91 |
| Claudiximab | 68.91 | 79.48 | 77.64 |

TM1: Melting temperature of Fab; TM2: Melting temperature of Fc (TM2 is not provided if TM1 and TM2 values are similar); TM_{agg}: Temperature where thermal aggregation stars.

### EQUIVALENTS/OTHER EMBODIMENTS

Those skilled in the art will recognize or be able to ascertain using no more than routine experimentation, many equivalents to the specific embodiments described herein. Such equivalents are intended to be encompassed by the following claims.

What is claimed is:

1. An antibody or antigen binding fragment thereof that is capable of binding to Claudin 18.2, comprising:
(a) (i) a heavy chain variable domain (VH) comprising complementarity-determining regions:
CDR-H1 comprising the amino acid sequence of SEQ ID NO: 2 or a sequence differing in 1 or 2 amino acids therefrom,
CDR-H2 comprising the amino acid sequence of SEQ ID NO: 3 or a sequence differing in 1 or 2 amino acids therefrom, and
CDR-H3 comprising the amino acid sequence of SEQ ID NO: 4 or a sequence differing in 1 or 2 amino acids therefrom; and
(ii) a light chain variable domain (VL) comprising complementarity-determining regions:
CDR-L1 comprising the amino acid sequence of SEQ ID NO: 6 or a sequence differing in 1 or 2 amino acids therefrom,
CDR-L2 comprising the amino acid sequence of SEQ ID NO: 7 or a sequence differing in 1 or 2 amino acids therefrom, and
CDR-L3 comprising the amino acid sequence of SEQ ID NO: 8 or a sequence differing in 1 or 2 amino acids therefrom:
(b) (i) a VH comprising complementarity-determining regions:
CDR-H1 comprising the amino acid sequence of SEQ ID NO: 10 or a sequence differing in 1 or 2 amino acids therefrom,
CDR-H2 comprising the amino acid sequence of SEQ ID NO: 11 or a sequence differing in 1 or 2 amino acids therefrom, and
CDR-H3 comprising the amino acid sequence of SEQ ID NO: 12 or a sequence differing in 1 or 2 amino acids therefrom; and
(ii) a VL comprising complementarity-determining regions:
CDR-L1 comprising the amino acid sequence of SEQ ID NO: 14 or a sequence differing in 1 or 2 amino acids therefrom,
CDR-L2 comprising the amino acid sequence of SEQ ID NO: 15 or a sequence differing in 1 or 2 amino acids therefrom, and
CDR-L3 comprising the amino acid sequence of SEQ ID NO: 16 or a sequence differing in 1 or 2 amino acids therefrom:
(c) (i) a VH comprising complementarity-determining regions:
CDR-H1 comprising the amino acid sequence of SEQ ID NO: 18 or a sequence differing in 1 or 2 amino acids therefrom,
CDR-H2 comprising the amino acid sequence of SEQ ID NO: 19 or a sequence differing in 1 or 2 amino acids therefrom, and
CDR-H3 comprising the amino acid sequence of SEQ ID NO: 20 or a sequence differing in 1 or 2 amino acids therefrom; and
(ii) a VL comprising complementarity-determining regions:
CDR-L1 comprising the amino acid sequence of SEQ ID NO: 22 or a sequence differing in 1 or 2 amino acids therefrom,
CDR-L2 comprising the amino acid sequence of SEQ ID NO: 23 or a sequence differing in 1 or 2 amino acids therefrom, and
CDR-L3 comprising the amino acid sequence of SEQ ID NO: 24 or a sequence differing in 1 or 2 amino acids therefrom:
(d) (i) a VH comprising complementarity-determining regions:
CDR-H1 comprising the amino acid sequence of SEQ ID NO: 26 or a sequence differing in 1 or 2 amino acids therefrom,
CDR-H2 comprising the amino acid sequence of SEQ ID NO: 27 or a sequence differing in 1 or 2 amino acids therefrom, and
CDR-H3 comprising the amino acid sequence of SEQ ID NO: 28 or a sequence differing in 1 or 2 amino acids therefrom; and
(ii) a VL comprising complementarity-determining regions:
CDR-L1 comprising the amino acid sequence of SEQ ID NO: 30 or a sequence differing in 1 or 2 amino acids therefrom,
CDR-L2 comprising the amino acid sequence of SEQ ID NO: 31 or a sequence differing in 1 or 2 amino acids therefrom, and
CDR-L3 comprising the amino acid sequence of SEQ ID NO: 32 or a sequence differing in 1 or 2 amino acids therefrom:
(e) (i) a VH comprising complementarity-determining regions:
CDR-H1 comprising the amino acid sequence of SEQ ID NO: 34 or a sequence differing in 1 or 2 amino acids therefrom,
CDR-H2 comprising the amino acid sequence of SEQ ID NO: 35 or a sequence differing in 1 or 2 amino acids therefrom, and
CDR-H3 comprising the amino acid sequence of SEQ ID NO: 36 or a sequence differing in 1 or 2 amino acids therefrom: and
(ii) a VL comprising complementarity-determining regions:
CDR-L1 comprising the amino acid sequence of SEQ ID NO: 38 or a sequence differing in 1 or 2 amino acids therefrom,
CDR-L2 comprising the amino acid sequence of SEQ ID NO: 39 or a sequence differing in 1 or 2 amino acids therefrom, and
CDR-L3 comprising the amino acid sequence of SEQ ID NO: 40 or a sequence differing in 1 or 2 amino acids therefrom:
(f) (i) a VH comprising complementarity-determining regions:
CDR-H1 comprising the amino acid sequence of SEQ ID NO: 42 or a sequence differing in 1 or 2 amino acids therefrom,
CDR-H2 comprising the amino acid sequence of SEQ ID NO: 43 or a sequence differing in 1 or 2 amino acids therefrom, and
CDR-H3 comprising the amino acid sequence of SEQ ID NO: 44 or a sequence differing in 1 or 2 amino acids therefrom; and
(ii) a VL comprising complementarity-determining regions:
CDR-L1 comprising the amino acid sequence of SEQ ID NO: 46 or a sequence differing in 1 or 2 amino acids therefrom,
CDR-L2 comprising the amino acid sequence of SEQ ID NO: 47 or a sequence differing in 1 or 2 amino acids therefrom, and
CDR-L3 comprising the amino acid sequence of SEQ ID NO: 48 or a sequence differing in 1 or 2 amino acids therefrom:
(g) (i) a VH comprising complementarity-determining regions:
CDR-H1 comprising the amino acid sequence of SEQ ID NO: 50 or a sequence differing in 1 or 2 amino acids therefrom,
CDR-H2 comprising the amino acid sequence of SEQ ID NO: 51 or a sequence differing in 1 or 2 amino acids therefrom, and
CDR-H3 comprising the amino acid sequence of SEQ ID NO: 52 or a sequence differing in 1 or 2 amino acids therefrom; and
(ii) a VL comprising complementarity-determining regions:
CDR-L1 comprising the amino acid sequence of SEQ ID NO: 54 or a sequence differing in 1 or 2 amino acids therefrom,
CDR-L2 comprising the amino acid sequence of SEQ ID NO: 55 or a sequence differing in 1 or 2 amino acids therefrom, and
CDR-L3 comprising the amino acid sequence of SEQ ID NO: 56 or a sequence differing in 1 or 2 amino acids therefrom:
(h) (i) a VH comprising complementarity-determining regions:
CDR-H1 comprising the amino acid sequence of SEQ ID NO: 58 or a sequence differing in 1 or 2 amino acids therefrom,
CDR-H2 comprising the amino acid sequence of SEQ ID NO: 59 or a sequence differing in 1 or 2 amino acids therefrom, and
CDR-H3 comprising the amino acid sequence of SEQ ID NO: 60 or a sequence differing in 1 or 2 amino acids therefrom: and
(ii) a VL comprising complementarity-determining regions:
CDR-L1 comprising the amino acid sequence of SEQ ID NO: 62 or a sequence differing in 1 or 2 amino acids therefrom,
CDR-L2 comprising the amino acid sequence of SEQ ID NO: 63 or a sequence differing in 1 or 2 amino acids therefrom, and
CDR-L3 comprising the amino acid sequence of SEQ ID NO: 64 or a sequence differing in 1 or 2 amino acids therefrom:
(i) (i) a VH comprising complementarity-determining regions:
CDR-H1 comprising the amino acid sequence of SEQ ID NO: 66 or a sequence differing in 1 or 2 amino acids therefrom,
CDR-H2 comprising the amino acid sequence of SEQ ID NO: 67 or a sequence differing in 1 or 2 amino acids therefrom, and
CDR-H3 comprising the amino acid sequence of SEQ ID NO: 68 or a sequence differing in 1 or 2 amino acids therefrom; and
(ii) a VL comprising complementarity-determining regions:
CDR-L1 comprising the amino acid sequence of SEQ ID NO: 70 or a sequence differing in 1 or 2 amino acids therefrom,
CDR-L2 comprising the amino acid sequence of SEQ ID NO: 71 or a sequence differing in 1 or 2 amino acids therefrom, and
CDR-L3 comprising the amino acid sequence of SEQ ID NO: 72 or a sequence differing in 1 or 2 amino acids therefrom:
(j) (i) a VH comprising complementarity-determining regions:
CDR-H1 comprising the amino acid sequence of SEQ ID NO: 74 or a sequence differing in 1 or 2 amino acids therefrom,
CDR-H2 comprising the amino acid sequence of SEQ ID NO: 75 or a sequence differing in 1 or 2 amino acids therefrom, and
CDR-H3 comprising the amino acid sequence of SEQ ID NO: 76 or a sequence differing in 1 or 2 amino acids therefrom; and
(ii) a VL comprising complementarity-determining regions:
CDR-L1 comprising the amino acid sequence of SEQ ID NO: 78 or a sequence differing in 1 or 2 amino acids therefrom,
CDR-L2 comprising the amino acid sequence of SEQ ID NO: 79 or a sequence differing in 1 or 2 amino acids therefrom, and
CDR-L3 comprising the amino acid sequence of SEQ ID NO: 80 or a sequence differing in 1 or 2 amino acids therefrom:
(k) (i) a VH comprising complementarity-determining regions:
CDR-H1 comprising the amino acid sequence of SEQ ID NO: 82 or a sequence differing in 1 or 2 amino acids therefrom,
CDR-H2 comprising the amino acid sequence of SEQ ID NO: 83 or a sequence differing in 1 or 2 amino acids therefrom, and
CDR-H3 comprising the amino acid sequence of SEQ ID NO: 84 or a sequence differing in 1 or 2 amino acids therefrom: and
(ii) a VL comprising complementarity-determining regions:
CDR-L1 comprising the amino acid sequence of SEQ ID NO: 86 or a sequence differing in 1 or 2 amino acids therefrom,
CDR-L2 comprising the amino acid sequence of SEQ ID NO: 87 or a sequence differing in 1 or 2 amino acids therefrom, and
CDR-L3 comprising the amino acid sequence of SEQ ID NO: 88 or a sequence differing in 1 or 2 amino acids therefrom:
(l) (i) a VH comprising complementarity-determining regions:
CDR-H1 comprising the amino acid sequence of SEQ ID NO: 90 or a sequence differing in 1 or 2 amino acids therefrom,
CDR-H2 comprising the amino acid sequence of SEQ ID NO: 91 or a sequence differing in 1 or 2 amino acids therefrom, and
CDR-H3 comprising the amino acid sequence of SEQ ID NO: 92 or a sequence differing in 1 or 2 amino acids therefrom; and
(ii) a VL comprising complementarity-determining regions:
CDR-L1 comprising the amino acid sequence of SEQ ID NO: 94 or a sequence differing in 1 or 2 amino acids therefrom,
CDR-L2 comprising the amino acid sequence of SEQ ID NO: 95 or a sequence differing in 1 or 2 amino acids therefrom, and
CDR-L3 comprising the amino acid sequence of SEQ ID NO: 96 or a sequence differing in 1 or 2 amino acids therefrom:
(m) (i) a VH comprising complementarity-determining regions:
CDR-H1 comprising the amino acid sequence of SEQ ID NO: 98 or a sequence differing in 1 or 2 amino acids therefrom,
CDR-H2 comprising the amino acid sequence of SEQ ID NO: 99 or a sequence differing in 1 or 2 amino acids therefrom, and
CDR-H3 comprising the amino acid sequence of SEQ ID NO: 100 or a sequence differing in 1 or 2 amino acids therefrom; and
(ii) a VL comprising complementarity-determining regions:
CDR-L1 comprising the amino acid sequence of SEQ ID NO: 102 or a sequence differing in 1 or 2 amino acids therefrom,
CDR-L2 comprising the amino acid sequence of SEQ ID NO: 103 or a sequence differing in 1 or 2 amino acids therefrom, and
CDR-L3 comprising the amino acid sequence of SEQ ID NO: 104 or a sequence differing in 1 or 2 amino acids therefrom: or
(n) (i) a VH comprising complementarity-determining regions:
CDR-H1 comprising the amino acid sequence of SEQ ID NO: 106 or a sequence differing in 1 or 2 amino acids therefrom,
CDR-H2 comprising the amino acid sequence of SEQ ID NO: 107 or a sequence differing in 1 or 2 amino acids therefrom, and
CDR-H3 comprising the amino acid sequence of SEQ ID NO: 108 or a sequence differing in 1 or 2 amino acids therefrom: and
(ii) a VL comprising complementarity-determining regions:
CDR-L1 comprising the amino acid sequence of SEQ ID NO: 110 or a sequence differing in 1 or 2 amino acids therefrom,
CDR-L2 comprising the amino acid sequence of SEQ ID NO: 111 or a sequence differing in 1 or 2 amino acids therefrom, and
CDR-L3 comprising the amino acid sequence of SEQ ID NO: 112 or a sequence differing in 1 or 2 amino acids therefrom.

2. The antibody or antigen fragment thereof of claim 1, wherein the CDR-H1, CDR-H2,CDR-H3, CDR-L1, CDR-L2, and CDR-L3 comprise amino acid sequences that collectively differ by no more than two amino acid residues from the sequences of:
(a) SEQ ID NO: 2, SEQ ID NO: 3, SEQ ID NO: 4, SEQ ID NO: 6, SEQ ID NO: 7,and SEQ ID NO: 8, respectively;
(b) SEQ ID NO: 10, SEQ ID NO: 11, SEQ ID NO: 12, SEQ ID NO: 14, SEQ ID NO: 15, and SEQ ID NO: 16, respectively;
(c) SEQ ID NO: 18, SEQ ID NO: 19, SEQ ID NO: 20, SEQ ID NO: 22, SEQ ID NO: 23, SEQ ID NO: 24, respectively;
(d) SEQ ID NO: 26, SEQ ID NO: 27, SEQ ID NO: 28, SEQ ID NO: 30, SEQ ID NO: 31, SEQ ID NO: 32, respectively;
(e) SEQ ID NO: 34, SEQ ID NO: 35, SEQ ID NO: 36, SEQ ID NO: 38, SEQ ID NO: 39, SEQ ID NO: 40, respectively;
(f) SEQ ID NO: 42, SEQ ID NO: 43, SEQ ID NO: 44, SEQ ID NO: 46, SEQ ID NO: 47, SEQ ID NO: 48, respectively;
(g) SEQ ID NO: 50, SEQ ID NO: 51, SEQ ID NO: 52, SEQ ID NO: 54, SEQ ID NO: 55, SEQ ID NO: 56, respectively;
(h) SEQ ID NO: 58, SEQ ID NO: 59, SEQ ID NO: 60, SEQ ID NO: 62, SEQ ID NO: 63, SEQ ID NO: 64, respectively;
(i) SEQ ID NO: 66, SEQ ID NO: 67, SEQ ID NO: 68, SEQ ID NO: 70, SEQ ID NO: 71, SEQ ID NO: 72, respectively;
(j) SEQ ID NO: 74, SEQ ID NO: 75, SEQ ID NO: 76, SEQ ID NO: 78, SEQ ID NO: 79, SEQ ID NO: 80, respectively;
(k) SEQ ID NO: 82, SEQ ID NO: 83, SEQ ID NO: 84, SEQ ID NO: 86, SEQ ID NO: 87, SEQ ID NO: 88, respectively;
(l) SEQ ID NO: 90, SEQ ID NO: 91, SEQ ID NO: 92, SEQ ID NO: 94, SEQ ID NO: 95, SEQ ID NO: 96, respectively;
(m) SEQ ID NO: 98, SEQ ID NO: 99, SEQ ID NO: 100, SEQ ID NO: 102, SEQ ID NO: 103, SEQ ID NO: 104, respectively;
or
(n) SEQ ID NO: 106, SEQ ID NO: 107, SEQ ID NO: 108, SEQ ID NO: 110, SEQ ID NO: 111, SEQ ID NO: 112, respectively.

3. The antibody or antigen binding fragment thereof of claim 2, comprising:
(a) (i) a VH comprising complementarity-determining regions:
CDR-H1 comprising the amino acid sequence of SEQ ID NO: 2,
CDR-H2 comprising the amino acid sequence of SEQ ID NO: 3, and
CDR-H3 comprising the amino acid sequence of SEQ ID NO: 4; and
(ii) a VL comprising complementarity-determining regions:
CDR-L1 comprising the amino acid sequence of SEQ ID NO: 6,
CDR-L2 comprising the amino acid sequence of SEQ ID NO: 7, and
CDR-L3 comprising the amino acid sequence of SEQ ID NO: 8:
(b) (i) a VH comprising complementarity-determining regions:
CDR-H1 comprising the amino acid sequence of SEQ ID NO: 10,
CDR-H2 comprising the amino acid sequence of SEQ ID NO: 11, and
CDR-H3 comprising the amino acid sequence of SEQ ID NO: 12; and
(ii) a VL comprising complementarity-determining regions:
CDR-L1 comprising the amino acid sequence of SEQ ID NO: 14,
CDR-L2 comprising the amino acid sequence of SEQ ID NO: 15, and
CDR-L3 comprising the amino acid sequence of SEQ ID NO: 16;
(c) (i) a VH comprising complementarity-determining regions:
CDR-H1 comprising the amino acid sequence of SEQ ID NO: 18,
CDR-H2 comprising the amino acid sequence of SEQ ID NO: 19, and
CDR-H3 comprising the amino acid sequence of SEQ ID NO: 20; and
(ii) a VL comprising complementarity-determining regions:
CDR-L1 comprising the amino acid sequence of SEQ ID NO: 22,
CDR-L2 comprising the amino acid sequence of SEQ ID NO: 23, and
CDR-L3 comprising the amino acid sequence of SEQ ID NO: 24;
(d) (i) a VH comprising complementarity-determining regions:
CDR-H1 comprising the amino acid sequence of SEQ ID NO: 26,
CDR-H2 comprising the amino acid sequence of SEQ ID NO: 27, and
CDR-H3 comprising the amino acid sequence of SEQ ID NO: 28; and
(ii) a VL comprising complementarity-determining regions:
CDR-L1 comprising the amino acid sequence of SEQ ID NO: 30,
CDR-L2 comprising the amino acid sequence of SEQ ID NO: 31, and
CDR-L3 comprising the amino acid sequence of SEQ ID NO: 32;
(e) (i) a VH comprising complementarity-determining regions:
CDR-H1 comprising the amino acid sequence of SEQ ID NO: 34,
CDR-H2 comprising the amino acid sequence of SEQ ID NO: 35, and
CDR-H3 comprising the amino acid sequence of SEQ ID NO: 36; and
(ii) a VL comprising complementarity-determining regions:
CDR-L1 comprising the amino acid sequence of SEQ ID NO: 38,
CDR-L2 comprising the amino acid sequence of SEQ ID NO: 39, and
CDR-L3 comprising the amino acid sequence of SEQ ID NO: 40;
(f) (i) a VH comprising complementarity-determining regions:
CDR-H1 comprising the amino acid sequence of SEQ ID NO: 42,
CDR-H2 comprising the amino acid sequence of SEQ ID NO: 43, and
CDR-H3 comprising the amino acid sequence of SEQ ID NO: 44: and
(ii) a VL comprising complementarity-determining regions:
CDR-L1 comprising the amino acid sequence of SEQ ID NO: 46,
CDR-L2 comprising the amino acid sequence of SEQ ID NO: 47, and
CDR-L3 comprising the amino acid sequence of SEQ ID NO: 48;
(g) (i) a VH comprising complementarity-determining regions:
CDR-H1 comprising the amino acid sequence of SEQ ID NO: 50,
CDR-H2 comprising the amino acid sequence of SEQ ID NO: 51, and
CDR-H3 comprising the amino acid sequence of SEQ ID NO: 52: and
(ii) a VL comprising complementarity-determining regions:
CDR-L1 comprising the amino acid sequence of SEQ ID NO: 54,
CDR-L2 comprising the amino acid sequence of SEQ ID NO: 55, and
CDR-L3 comprising the amino acid sequence of SEQ ID NO: 56;
(h) (i) a VH comprising complementarity-determining regions:
CDR-H1 comprising the amino acid sequence of SEQ ID NO: 58,
CDR-H2 comprising the amino acid sequence of SEQ ID NO: 59, and
CDR-H3 comprising the amino acid sequence of SEQ ID NO: 60: and
(ii) a VL comprising complementarity-determining regions:
CDR-L1 comprising the amino acid sequence of SEQ ID NO: 62,
CDR-L2 comprising the amino acid sequence of SEQ ID NO: 63, and
CDR-L3 comprising the amino acid sequence of SEQ ID NO: 64;
(i) (i) a VH comprising complementarity-determining regions:
CDR-H1 comprising the amino acid sequence of SEQ ID NO: 66,
CDR-H2 comprising the amino acid sequence of SEQ ID NO: 67, and
CDR-H3 comprising the amino acid sequence of SEQ ID NO: 68: and
(ii) a VL comprising complementarity-determining regions:
CDR-L1 comprising the amino acid sequence of SEQ ID NO: 70,
CDR-L2 comprising the amino acid sequence of SEQ ID NO: 71, and
CDR-L3 comprising the amino acid sequence of SEQ ID NO: 72:
(j) (i) a VH comprising complementarity-determining regions:
CDR-H1 comprising the amino acid sequence of SEQ ID NO: 74,
CDR-H2 comprising the amino acid sequence of SEQ ID NO: 75, and
CDR-H3 comprising the amino acid sequence of SEQ ID NO: 76: and
(ii) a VL comprising complementarity-determining regions:
CDR-L1 comprising the amino acid sequence of SEQ ID NO: 78,
CDR-L2 comprising the amino acid sequence of SEQ ID NO: 79, and
CDR-L3 comprising the amino acid sequence of SEQ ID NO: 80;
(k) (i) a VH comprising complementarity-determining regions:
CDR-H1 comprising the amino acid sequence of SEQ ID NO: 82,
CDR-H2 comprising the amino acid sequence of SEQ ID NO: 83, and
CDR-H3 comprising the amino acid sequence of SEQ ID NO: 84; and
(ii) a VL comprising complementarity-determining regions:
CDR-L1 comprising the amino acid sequence of SEQ ID NO: 86,
CDR-L2 comprising the amino acid sequence of SEQ ID NO: 87, and
CDR-L3 comprising the amino acid sequence of SEQ ID NO: 88;
(l) (i) a VH comprising complementarity-determining regions:
CDR-H1 comprising the amino acid sequence of SEQ ID NO: 90,
CDR-H2 comprising the amino acid sequence of SEQ ID NO: 91, and
CDR-H3 comprising the amino acid sequence of SEQ ID NO: 92: and
(ii) a VL comprising complementarity-determining regions:
CDR-L1 comprising the amino acid sequence of SEQ ID NO: 94,
CDR-L2 comprising the amino acid sequence of SEQ ID NO: 95, and
CDR-L3 comprising the amino acid sequence of SEQ ID NO: 96;
(m) (i) a VH comprising complementarity-determining regions:
CDR-H1 comprising the amino acid sequence of SEQ ID NO: 98,
CDR-H2 comprising the amino acid sequence of SEQ ID NO: 99, and
CDR-H3 comprising the amino acid sequence of SEQ ID NO: 100; and
(ii) a VL comprising complementarity-determining regions:
CDR-L1 comprising the amino acid sequence of SEQ ID NO: 102,
CDR-L2 comprising the amino acid sequence of SEQ ID NO: 103, and
CDR-L3 comprising the amino acid sequence of SEQ ID NO: 104; or
(n) (i) a VH comprising complementarity-determining regions:
CDR-H1 comprising the amino acid sequence of SEQ ID NO: 106,
CDR-H2 comprising the amino acid sequence of SEQ ID NO: 107, and
CDR-H3 comprising the amino acid sequence of SEQ ID NO: 108; and
(ii) a VL comprising complementarity-determining regions:
CDR-L1 comprising the amino acid sequence of SEQ ID NO: 110,
CDR-L2 comprising the amino acid sequence of SEQ ID NO: 111, and
CDR-L3 comprising the amino acid sequence of SEQ ID NO: 112.

4. The antibody or antigen binding fragment thereof of claim 3, comprising:
(i) a VH comprising complementarity-determining regions:
CDR-H1 comprising the amino acid sequence of SEQ ID NO: 2;
CDR-H2 comprising the amino acid sequence of SEQ ID NO: 3; and
CDR-H3 comprising the amino acid sequence of SEQ ID NO: 4; and
(ii) a VL comprising complementarity-determining regions:
CDR-L1 comprising the amino acid sequence of SEQ ID NO: 6;
CDR-L2 comprising the amino acid sequence of SEQ ID NO: 7; and
CDR-L3 comprising the amino acid sequence of SEQ ID NO: 8.

5. The antibody or antigen binding fragment thereof of any one of claims 1-4, wherein:
the VH comprises an amino acid sequence that is at least 85% identical to that of SEQ ID NO: 1; and
the VL comprises an amino acid sequence that is at least 85% identical to that of SEQ ID NO: 5.

6. The antibody or antigen binding fragment thereof of claim 5, wherein:
the VH comprises the amino acid sequence of SEQ ID NO: 1; and
the VL comprises the amino acid sequence of SEQ ID NO: 5.

7. The antibody or antigen binding fragment thereof of claim 3, comprising:
(i) a VH comprising complementarity-determining regions:
CDR-H1 comprising the amino acid sequence of SEQ ID NO: 82;
CDR-H2 comprising the amino acid sequence of SEQ ID NO: 83; and
CDR-H3 comprising the amino acid sequence of SEQ ID NO: 84; and
(ii) a light chain variable domain (VL) comprising complementarity-determining regions:
CDR-L1 comprising the amino acid sequence of SEQ ID NO: 86;
CDR-L2 comprising the amino acid sequence of SEQ ID NO: 87; and
CDR-L3 comprising the amino acid sequence of SEQ ID NO: 88.

8. The antibody or antigen binding fragment thereof of any one of claim 1-3 or 7, wherein:
the VH comprises an amino acid sequence that is at least 85% identical to that of SEQ ID NO: 81; and
the VL comprises an amino acid sequence that is at least 85% identical to that of SEQ ID NO: 85.

9. The antibody or antigen binding fragment thereof of claim 8, wherein:
the VH comprises the amino acid sequence of SEQ ID NO: 81; and
the VL comprises the amino acid sequence of SEQ ID NO: 85.

10. The antibody or antigen binding fragment thereof of claim 1, wherein
(a) the VH comprises an amino acid sequence at least 90%, 95%, 96%, 97%, 98%, or 99% identical to SEQ ID NO: 1 and the VL comprises an amino acid sequence at least 90%, 95%, 96%, 97%, 98%, or 99% identical to SEQ ID NO: 5;
(b) the VH comprises an amino acid sequence at least 90%, 95%, 96%, 97%, 98%, or 99% identical to SEQ ID NO: 9 and the VL comprises an amino acid sequence at least 90%, 95%, 96%, 97%, 98%, or 99% identical to SEQ ID NO: 13;
(c) the VH comprises an amino acid sequence at least 90%, 95%, 96%, 97%, 98%, or 99% identical to SEQ ID NO: 17 and the VL comprises an amino acid sequence at least 90%, 95%, 96%, 97%, 98%, or 99% identical to SEQ ID NO: 21;
(d) the VH comprises an amino acid sequence at least 90%, 95%, 96%, 97%, 98%, or 99% identical to SEQ ID NO: 25 and the VL comprises an amino acid sequence at least 90%, 95%, 96%, 97%, 98%, or 99% identical to SEQ ID NO: 29;
(e) the VH comprises an amino acid sequence at least 90%, 95%, 96%, 97%, 98%, or 99% identical to SEQ ID NO: 33 and the VL comprises an amino acid sequence at least 90%, 95%, 96%, 97%, 98%, or 99% identical to SEQ ID NO: 37;
(f) the VH comprises an amino acid sequence at least 90%, 95%, 96%, 97%, 98%, or 99% identical to SEQ ID NO: 41 and the VL comprises an amino acid sequence at least 90%, 95%, 96%, 97%, 98%, or 99% identical to SEQ ID NO: 45;
(g) the VH comprises an amino acid sequence at least 90%, 95%, 96%, 97%, 98%, or 99% identical to SEQ ID NO: 49 and the VL comprises an amino acid sequence at least 90%, 95%, 96%, 97%, 98%, or 99% identical to SEQ ID NO: 53;
(h) the VH comprises an amino acid sequence at least 90%, 95%, 96%, 97%, 98%, or 99% identical to SEQ ID NO: 57 and the VL comprises an amino acid sequence at least 90%, 95%, 96%, 97%, 98%, or 99% identical to SEQ ID NO: 61;
(i) the VH comprises an amino acid sequence at least 90%, 95%, 96%, 97%, 98%, or 99% identical to SEQ ID NO: 65 and the VL comprises an amino acid sequence at least 90%, 95%, 96%, 97%, 98%, or 99% identical to SEQ ID NO: 69;
(j) the VH comprises an amino acid sequence at least 90%, 95%, 96%, 97%, 98%, or 99% identical to SEQ ID NO: 73 and the VL comprises an amino acid sequence at least 90%, 95%, 96%, 97%, 98%, or 99% identical to SEQ ID NO: 77;
(k) the VH comprises an amino acid sequence at least 90%, 95%, 96%, 97%, 98%, or 99% identical to SEQ ID NO: 81 and the VL comprises an amino acid sequence at least 90%, 95%, 96%, 97%, 98%, or 99% identical to SEQ ID NO: 85;
(l) the VH comprises an amino acid sequence at least 90%, 95%, 96%, 97%, 98%, or 99% identical to SEQ ID NO: 89 and the VL comprises an amino acid sequence at least 90%, 95%, 96%, 97%, 98%, or 99% identical to SEQ ID NO: 93;
(m) the VH comprises an amino acid sequence at least 90%, 95%, 96%, 97%, 98%, or 99% identical to SEQ ID NO: 97 and the VL comprises an amino acid sequence at least 90%, 95%, 96%, 97%, 98%, or 99% identical to SEQ ID NO: 101; or
(n) the VH comprises an amino acid sequence at least 90%, 95%, 96%, 97%, 98%, or 99% identical to SEQ ID NO: 105 and the VL comprises an amino acid sequence at least 90%, 95%, 96%, 97%, 98%, or 99% identical to SEQ ID NO: 109.

11. The antibody or antigen binding fragment thereof of claim 1, wherein
(a) the VH comprises an amino acid sequence of SEQ ID NO: 1 and the VL comprises an amino acid sequence of SEQ ID NO: 5;
(b) the VH comprises an amino acid sequence of SEQ ID NO: 9 and the VL comprises an amino acid sequence of SEQ ID NO: 13;
(c) the VH comprises an amino acid sequence of SEQ ID NO: 17 and the VL comprises an amino acid sequence of SEQ ID NO: 21;
(d) the VH comprises an amino acid sequence of SEQ ID NO: 25 and the VL comprises an amino acid sequence of SEQ ID NO: 29;
(e) the VH comprises an amino acid sequence of SEQ ID NO: 33 and the VL comprises an amino acid sequence of SEQ ID NO: 37;
(f) the VH comprises an amino acid sequence of SEQ ID NO: 41 and the VL comprises an amino acid sequence of SEQ ID NO: 45;
(g) the VH comprises an amino acid sequence of SEQ ID NO: 49 and the VL comprises an amino acid sequence of SEQ ID NO: 53;
(h) the VH comprises an amino acid sequence of SEQ ID NO: 57 and the VL comprises an amino acid sequence of SEQ ID NO: 61;
(i) the VH comprises an amino acid sequence of SEQ ID NO: 65 and the VL comprises an amino acid sequence of SEQ ID NO: 69;
(j) the VH comprises an amino acid sequence of SEQ ID NO: 73 and the VL comprises an amino acid sequence of SEQ ID NO: 77;
(k) the VH comprises an amino acid sequence of SEQ ID NO: 81 and the VL comprises an amino acid sequence of SEQ ID NO: 85;
(l) the VH comprises an amino acid sequence of SEQ ID NO: 89 and the VL comprises an amino acid sequence of SEQ ID NO: 93;
(m) the VH comprises an amino acid sequence of SEQ ID NO: 97 and the VL comprises an amino acid sequence of SEQ ID NO: 101; or
(n) the VH comprises an amino acid sequence of SEQ ID NO: 105 and the VL comprises an amino acid sequence of SEQ ID NO: 109.

12. The antibody or antigen binding fragment thereof of any one of the preceding claims, wherein the antibody or antigen binding fragment thereof is capable of binding to human Claudin 18.2 with an EC₅₀ value of at most about 10 nM or lower, at most about 8 nM or lower, at most about 6 nM or lower, at most about 5 nM or lower, at most about 4 nM or lower, or at most about 3 nM or lower, wherein the EC₅₀ value is determined by a flow cytometry assay as described in Example 4.

13. The antibody or antigen binding fragment thereof of any one of the preceding claims, wherein the antibody or antigen binding fragment thereof is capable of binding to mouse Claudin 18.2.

14. The antibody or antigen binding fragment thereof of any one of the preceding claims, wherein the antibody or antigen binding fragment thereof is not capable of significantly binding to Claudin 18.1.

15. The antibody or antigen binding fragment thereof of any one of the preceding claims, wherein the antibody or antigen binding fragment thereof is capable of being internalized after contacting with cells expressing Claudin 18.2.

16. The antibody or antigen binding fragment thereof of any one of the preceding claims, wherein the antibody is a human antibody.

17. The antibody or antigen binding fragment thereof of any one of the preceding claims, wherein the antigen binding fragment is a Fab, a F(ab)₂, a Fab′, a single-chain Fv (scFv), an Fv fragment, a Fd fragment, or a diabody.

18. The antibody or antigen binding fragment thereof of any one of the preceding claims, wherein the antigen binding fragment is an scFv.

19. The antibody or antigen binding fragment thereof of any one of the preceding claims, wherein the antibody or antigen binding fragment thereof comprises an antibody heavy chain constant region.

20. The antibody or antigen binding fragment thereof of claim 19, wherein the heavy chain constant region is a human IgG heavy chain constant region.

21. The antibody or antigen binding fragment thereof of claim 19 or 20, wherein the antibody heavy chain constant region is a human IgG4 heavy chain constant region.

22. The antibody or antigen binding fragment thereof of claim 21, wherein the human IgG4 heavy chain constant region comprises a S228P mutation.

23. The antibody or antigen binding fragment thereof of any one of the preceding claims, conjugated to a therapeutic agent.

24. The antibody or antigen binding fragment thereof of any one of the preceding claims, conjugated to a label.

25. An isolated nucleic acid encoding an antibody or antigen binding fragment thereof of any one of the preceding claims.

26. An expression vector comprising the isolated nucleic acid of claim 25.

27. A host cell comprising the isolated nucleic acid molecule of claim 25 or the expression vector of claim 26.

28. A composition comprising an antibody or antigen binding fragment thereof of any one of claims 1-22 or antibody conjugate of claim 23 or 24 and a pharmaceutically acceptable carrier.

29. The composition of claim 28, further comprising a therapeutic agent.

30. A method of treating cancer in a subject, comprising administering to the subject an effective amount of the antibody or antigen binding fragment thereof of any one of claims 1-22 or antibody conjugate of claim 23 or 24.

31. A method of detecting the presence of Claudin 18.2, or a fragment thereof, in a sample, comprising contacting the sample with an antibody or antigen binding fragment of any of claims 1-22 and detecting the presence of a complex between the antibody or antigen binding fragment and Claudin 18.2, wherein detection of the complex indicates the presence of Claudin 18.2.


SEQUENCE LISTING (112 sequences)

SEQ ID NO: 1 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAGRQVAWLGNWGQGTLVTVSS

SEQ ID NO: 2 - protein, 5 aa, synthetic construct
SYAMS

SEQ ID NO: 3 - protein, 17 aa, synthetic construct
AISGSGGSTYYADSVKG

SEQ ID NO: 4 - protein, 8 aa, synthetic construct
RQVAWLGN

SEQ ID NO: 5 - protein, 107 aa, synthetic construct
DIVMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKVDIK

SEQ ID NO: 6 - protein, 11 aa, synthetic construct
RASQGISSWLA

SEQ ID NO: 7 - protein, 7 aa, synthetic construct
AASSLQS

SEQ ID NO: 8 - protein, 9 aa, synthetic construct
QQANSFPLT

SEQ ID NO: 9 - protein, 116 aa, synthetic construct
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAGGANGFDPWGQGTLVTVSS

SEQ ID NO: 10 - protein, 5 aa, synthetic construct
SYAMS

SEQ ID NO: 11 - protein, 17 aa, synthetic construct
AISGSGGSTYYADSVKG

SEQ ID NO: 12 - protein, 7 aa, synthetic construct
GANGFDP

SEQ ID NO: 13 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGLVFGGGTKLTVL

SEQ ID NO: 14 - protein, 14 aa, synthetic construct
TGSSSNIGAGYDVH

SEQ ID NO: 15 - protein, 7 aa, synthetic construct
GNSNRPS

SEQ ID NO: 16 - protein, 11 aa, synthetic construct
QSYDSSLSGLV

SEQ ID NO: 17 - protein, 120 aa, synthetic construct
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCAKDQRGGLYGMDVWGQGTLVTVSS

SEQ ID NO: 18 - protein, 5 aa, synthetic construct
SYGMH

SEQ ID NO: 19 - protein, 17 aa, synthetic construct
VISYDGSNKYYADSVKG

SEQ ID NO: 20 - protein, 11 aa, synthetic construct
DQRGGLYGMDV

SEQ ID NO: 21 - protein, 111 aa, synthetic construct
QSVLTQPPSASGAPGQRVTISCTGSSSNIGAGYDVHWYQQLPGTAPKLLIYGNSNRPSGVPDRFSGSKSGTSASLAITGLQAEDEADYYCQSYDSSLSGWVFGGGTKLTVL

SEQ ID NO: 22 - protein, 14 aa, synthetic construct
TGSSSNIGAGYDVH

SEQ ID NO: 23 - protein, 7 aa, synthetic construct
GNSNRPS

SEQ ID NO: 24 - protein, 11 aa, synthetic construct
QSYDSSLSGWV

SEQ ID NO: 25 - protein, 116 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGGSGFDYWGQGTLVTVSS

SEQ ID NO: 26 - protein, 5 aa, synthetic construct
SYYMH

SEQ ID NO: 27 - protein, 17 aa, synthetic construct
IINPSGGSTSYAQKFQG

SEQ ID NO: 28 - protein, 7 aa, synthetic construct
GGSGFDY

SEQ ID NO: 29 - protein, 110 aa, synthetic construct
SYVLTQPPSASGTPGQRVTISCSGSSSNIGSNYVYWYQQLPGTAPKLLIYSNNQRPSGVPDRFSGSKSGTSASLAISGLRSEDEADYYCAAWDDSLSGWVFGGGTKLTVL

SEQ ID NO: 30 - protein, 13 aa, synthetic construct
SGSSSNIGSNYVY

SEQ ID NO: 31 - protein, 7 aa, synthetic construct
SNNQRPS

SEQ ID NO: 32 - protein, 11 aa, synthetic construct
AAWDDSLSGWV

SEQ ID NO: 33 - protein, 118 aa, synthetic construct
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAIPGDYGSSDPWGQGTLVTVSS

SEQ ID NO: 34 - protein, 5 aa, synthetic construct
SYAMS

SEQ ID NO: 35 - protein, 17 aa, synthetic construct
AISGSGGSTYYADSVKG

SEQ ID NO: 36 - protein, 9 aa, synthetic construct
PGDYGSSDP

SEQ ID NO: 37 - protein, 111 aa, synthetic construct
QSVLTQPPSVSGAPGQRVTISCTGSSSNIGAGYDVHWYQQLPGTAPKLLIYGNSNRPSGVPDRFSGSKSGTSASLAITGLQAEDEADYYCQSYDSSLSGYVFGTGTKLTVL

SEQ ID NO: 38 - protein, 14 aa, synthetic construct
TGSSSNIGAGYDVH

SEQ ID NO: 39 - protein, 7 aa, synthetic construct
GNSNRPS

SEQ ID NO: 40 - protein, 11 aa, synthetic construct
QSYDSSLSGYV

SEQ ID NO: 41 - protein, 125 aa, synthetic construct
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKDVTYYYDSSGSPDFDYWGQGTLVTVSS

SEQ ID NO: 42 - protein, 5 aa, synthetic construct
SYGMH

SEQ ID NO: 43 - protein, 17 aa, synthetic construct
VISYDGSNKYYADSVKG

SEQ ID NO: 44 - protein, 16 aa, synthetic construct
DVTYYYDSSGSPDFDY

SEQ ID NO: 45 - protein, 107 aa, synthetic construct
DIQLTQSPSSLSASVGDRVTITCRASQGISNYLAWFQQKPGKAPKSLIYAASSLQSGVPSKFSGSGSGTDFTLTISSLQPEDFATYYCQQYNSYPYSFGQGTKVEIK

SEQ ID NO: 46 - protein, 11 aa, synthetic construct
RASQGISNYLA

SEQ ID NO: 47 - protein, 7 aa, synthetic construct
AASSLQS

SEQ ID NO: 48 - protein, 9 aa, synthetic construct
QQYNSYPYS

SEQ ID NO: 49 - protein, 117 aa, synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAGYTGGAFDIWGQGTMVTVSS

SEQ ID NO: 50 - protein, 5 aa, synthetic construct
SYAMS

SEQ ID NO: 51 - protein, 17 aa, synthetic construct
AISGSGGSTYYADSVKG

SEQ ID NO: 52 - protein, 8 aa, synthetic construct
YTGGAFDI

SEQ ID NO: 53 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQANSFPLTFGGGTKLEIK

SEQ ID NO: 54 - protein, 11 aa, synthetic construct
RASQGISSWLA

SEQ ID NO: 55 - protein, 7 aa, synthetic construct
AASSLQS

SEQ ID NO: 56 - protein, 9 aa, synthetic construct
QQANSFPLT

SEQ ID NO: 57 - protein, 119 aa, synthetic construct
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKPLRFLGDYGGWGQGTLVTVSS

SEQ ID NO: 58 - protein, 5 aa, synthetic construct
SYAMS

SEQ ID NO: 59 - protein, 17 aa, synthetic construct
AISGSGGSTYYADSVKG

SEQ ID NO: 60 - protein, 10 aa, synthetic construct
PLRFLGDYGG

SEQ ID NO: 61 - protein, 107 aa, synthetic construct
DIQMTQSPSSLSASVGDRVTITCQASQDISNYLNWYQQKPGKAPKLLIYDASNLETGVPSRFSGSGSGTDFTFTISSLQPEDIATYYCQQYDNLPLTFGGGTKLEIK

SEQ ID NO: 62 - protein, 11 aa, synthetic construct
QASQDISNYLN

SEQ ID NO: 63 - protein, 7 aa, synthetic construct
DASNLET

SEQ ID NO: 64 - protein, 9 aa, synthetic construct
QQYDNLPLT

SEQ ID NO: 65 - protein, 114 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCATRQFDYWGQGTLVTVSS

SEQ ID NO: 66 - protein, 5 aa, synthetic construct
SYYMH

SEQ ID NO: 67 - protein, 17 aa, synthetic construct
IINPSGGSTSYAQKFQG

SEQ ID NO: 68 - protein, 5 aa, synthetic construct
RQFDY

SEQ ID NO: 69 - protein, 110 aa, synthetic construct
QSVLTQPPSVSAAPGQKVTISCSGSSSNIGNNYVSWYQQLPGTAPKLLIYENNKRPSGIPDRFSGSKSGTSATLGITGLQTGDEADYYCGTWDSSLSAAVFGGGTKVTVL

SEQ ID NO: 70 - protein, 13 aa, synthetic construct
SGSSSNIGNNYVS

SEQ ID NO: 71 - protein, 7 aa, synthetic construct
ENNKRPS

SEQ ID NO: 72 - protein, 11 aa, synthetic construct
GTWDSSLSAAV

SEQ ID NO: 73 - protein, 118 aa, synthetic construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLGVYYFDYWGQGTLVTVSS

SEQ ID NO: 74 - protein, 5 aa, synthetic construct
SYAMS

SEQ ID NO: 75 - protein, 17 aa, synthetic construct
AISGSGGSTYYADSVKG

SEQ ID NO: 76 - protein, 9 aa, synthetic construct
DLGVYYFDY

SEQ ID NO: 77 - protein, 107 aa, synthetic construct
DIVMTQSPSFLSASAGDRVTITCRASQGISSYLAWYQQKPGKGPKLLIYAASTLQSGVPSRFSGSGSGTEFTLTISSLQPEDFATYYCQQVNSYPLTFGPGTKVDIK

SEQ ID NO: 78 - protein, 11 aa, synthetic construct
RASQGISSYLA

SEQ ID NO: 79 - protein, 7 aa, synthetic construct
AASTLQS

SEQ ID NO: 80 - protein, 9 aa, synthetic construct
QQVNSYPLT

SEQ ID NO: 81 - protein, 117 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKEPFYGMDVWGQGTLVTVSS

SEQ ID NO: 82 - protein, 5 aa, synthetic construct
SYAMS

SEQ ID NO: 83 - protein, 17 aa, synthetic construct
AISGSGGSTYYADSVKG

SEQ ID NO: 84 - protein, 8 aa, synthetic construct
EPFYGMDV

SEQ ID NO: 85 - protein, 107 aa, synthetic construct; identical to SEQ ID NO: 53

SEQ ID NO: 86 - protein, 11 aa, synthetic construct
RASQGISSWLA

SEQ ID NO: 87 - protein, 7 aa, synthetic construct
AASSLQS

SEQ ID NO: 88 - protein, 9 aa, synthetic construct
QQANSFPLT

SEQ ID NO: 89 - protein, 119 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKPPGSYYVWDYWGQGTLVTVSS

SEQ ID NO: 90 - protein, 5 aa, synthetic construct
SYAMS

SEQ ID NO: 91 - protein, 17 aa, synthetic construct
AISGSGGSTYYADSVKG

SEQ ID NO: 92 - protein, 10 aa, synthetic construct
PPGSYYVWDY

SEQ ID NO: 93 - protein, 107 aa, synthetic construct
DIVMTQSPSTLSASVGDRVTITCRASQSISSWLAWYQQKPGKAPKLLIYKASSLESGVPSRFSGSGSGTEFTLTISSLQPDDFATYYCQQYNSYPLTFGGGTKVEIK

SEQ ID NO: 94 - protein, 11 aa, synthetic construct
RASQSISSWLA

SEQ ID NO: 95 - protein, 7 aa, synthetic construct
KASSLES

SEQ ID NO: 96 - protein, 9 aa, synthetic construct
QQYNSYPLT

SEQ ID NO: 97 - protein, 119 aa, synthetic construct
EVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKTAGSYYILDYWGQGTLVTVSS

SEQ ID NO: 98 - protein, 5 aa, synthetic construct
SYAMS

SEQ ID NO: 99 - protein, 17 aa, synthetic construct
AISGSGGSTYYADSVKG

SEQ ID NO: 100 - protein, 10 aa, synthetic construct
TAGSYYILDY

SEQ ID NO: 101 - protein, 107 aa, synthetic construct
DIQLTQSPSSLSASVGDRVTITCRASQGISNYLAWFQQKPGKAPKSLIYAASSLQSGVPSKFSGSGSGTDFTLTISSLQPEDFATYYCQQYNSYPLTFGGGTKVDIK

SEQ ID NO: 102 - protein, 11 aa, synthetic construct
RASQGISNYLA

SEQ ID NO: 103 - protein, 7 aa, synthetic construct
AASSLQS

SEQ ID NO: 104 - protein, 9 aa, synthetic construct
QQYNSYPLT

SEQ ID NO: 105 - protein, 124 aa, synthetic construct
QVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKPLTAGYYYGMVAFDIWGQGTMVTVSS

SEQ ID NO: 106 - protein, 5 aa, synthetic construct
SYAMS

SEQ ID NO: 107 - protein, 17 aa, synthetic construct
AISGSGGSTYYADSVKG

SEQ ID NO: 108 - protein, 15 aa, synthetic construct
PLTAGYYYGMVAFDI

SEQ ID NO: 109 - protein, 107 aa, synthetic construct
DIVMTQSPSFLSASVGDRVTITCRASQGISSYLAWYQQKPGKAPKLLIYAASTLQSGVPSRFSGSGSGTEFTLTISSLQPEDFATYYCQQLNSYPITFGQGTKLEIK

SEQ ID NO: 110 - protein, 11 aa, synthetic construct
RASQGISSYLA

SEQ ID NO: 111 - protein, 7 aa, synthetic construct
AASTLQS

SEQ ID NO: 112 - protein, 9 aa, synthetic construct
QQLNSYPIT